(19) United States
(12) Patent Application Publication
(10) Pub. No.: US 2002/0127543 A1
(43) Pub. Date: Sep. 12, 2002

(54) Methods and compositions utilizing hepatitis C virus molecules

(76) Inventors: Jonathan Karn, Cambridge (GB); Stephen Walker, Willingham (GB)

Correspondence Address: KATHLEEN M. WILLIAMS, PH.D, Palmer & Dodge, LLP, One Beacon Street, Boston, MA, 02108, US

(21) Appl. No.: 09/746,170

(22) Filed: Dec. 22, 2000

Related U.S. Application Data
(60) Provisional application No. 60/171,804, filed on Dec. 22, 1999.

(51) Int. Cl. C12Q 1/70; G01N 33/53; C07H 21/00; C12Q 1/68; C07H 21/04; C07H 21/02

(52) U.S. Cl. 435/5; 536/23.72; 536/23.2; 536/25.32; 435/6; 435/7.1

(57) ABSTRACT

The invention provides for compounds comprising nucleotide sequences 5′-P-N¹-N²-G-N³-C-I-3′ and 3′-Q-N⁴-N⁵-N⁶-N⁷-N⁸-N⁹-Y-J-5′ that are the generic sequences of a mIRES of an HCV genome, and variants thereof The invention also provides assays that utilise the compounds of the invention, including assays for detection of HCV antiviral compounds.

### TECHNICAL FIELD OF THE INVENTION

This invention relates to assays for molecules that interact with the hepatitis C virus genome and to compounds for use in such assays.

### BACKGROUND OF THE INVENTION

Cap-independent translation of hepatitis C virus (HCV) genomic RNA is mediated by an internal ribosome entry site (IRES) within the 5′-UTR of the viral RNA (FIG. 1), and inhibiting the interaction of translation initiation factors (e.g. Eukaryotic Initiation Factor 3-eIF3) with the 5′UTR has been proposed as a therapeutic strategy [e.g. references i, ii and iii].

Assays for this inhibition are not, however, straightforward. Culture of HCV is very difficult and slow, so indirect assays and cell-free systems are used instead, in which translation of a reporter gene controlled by the 5′-UTR is monitored. Several reporter genes have been described. Reference iv utilises the chloramphenicol acetyltransferase (CAT) gene fused to the HCV-1b 5′-UTR, with a subsequent assay of CAT activity indicating whether translation from the 5′-UTR was inhibited. Reference v describes a bicistronic construct containing two different luciferases, the first being translated in a cap-dependent manner and the second being translated, like wild-type HCV, in a cap-independent manner. The relative levels of the two luciferases gives an indication of whether the cap-independent translation was inhibited. By determining cap-independent translation in the present of test molecules, therefore, these systems offer indirect ways of following the prevention of translation initiation in the 5′-UTR.

Whilst these indirect assays are easier than direct testing of HCV in culture, they still have drawbacks. Firstly, the complete 5′-UTR is required in each case. Secondly, they rely on protein synthesis and therefore on the presence of ribosomes and free amino acids in the system, making the assays relatively cumbersome, time-consuming and expensive. Finally, indirect assays cannot, by their nature, directly monitor the eIF3/5′-UTR interaction. Positive results may, therefore, reflect an interaction of the test molecule with the ribosome or with a host protein required for IRES function, rather than specific inhibition of the 5′-UTR itself.

There is thus a need for simpler, quicker and more cost effective assays for monitoring 5′-UTR activity.

Specific binding of eIF3 to the HCV IRES has been reported by Sizova et al. [vi]. Sizova et al. reported an enzymatic footprinting analysis of a complex formed between eIF3 and the complete HCV IRES. In particular, eIF3 was found to protect certain domains of the HCV IRES from cleavage by RNases ONE and V₁. Such nuclease footprints may arise for a number of reasons. For example, inhibition of nuclease cleavage of the IRES may be the result of eIF3 binding at a site in the IRES which induces a conformational change in the IRES; or it may be the result of a steric shadow arising from binding of eIF3 at an adjacent or remote site in the IRES; or it may be the result of direct interaction of eIF3 with the binding site; or it may be the result of an artefact of the footprinting technique.

Kieft et al. [vii] report the results of mutagenesis experiments in which point mutations were made at the IIIabc four-way junction and in the IIId loop. The authors report that one mutation (U228C) in the IIIabc junction results in >95% inhibition of HCV IRES activity and that the IIId loop was critical for function.

### SUMMARY

A small sub-region of the 5′-UTR of HCV RNA, shown in generalised formula in FIG. 2, is essential for the binding of eIF3. This sub-region can be used in an assay to assist in the identification of molecules or compounds (for example, drugs) which inhibit HCV translation initiation. Assays based on this sub-region, referred to hereafter as ‘mIRES’ (for ‘minimal IRES’) enable potential anti-viral molecules to be screened more efficiently and in a more cost effective manner. The compounds of the invention, which comprise the mIRES or mimics thereof, allow rapid assays using a small volume of material, which are also suited to parallel processing.

According to the present invention there is provided a compound comprising nucleotide sequences:

5′-P-N¹-N²-G-N³-C-I-3′ and 3′-Q-N⁴-N⁵-N⁶-N⁷-N⁸-N⁹-Y-J-5′

wherein

P and Q are any two nucleotides that can form a Watson-Crick base pair,

I and J are any two nucleotides that can form a base pair, preferably a Watson-Crick base pair,

N¹ and N⁴ are not both C,

when N²is A, N⁷is not G,

N⁹ can only be U if N³ is A,

when N³ is G, N⁹ is A,

the sequences can anneal to each other, and the compound comprises 200 or fewer nucleotides.

As used herein, a “base pair” refers to two nucleotides in a nucleic acid molecule (i.e., RNA or DNA) that are associated by hydrogen bonds. For example, a nucleotide comprising a guanine base hydrogen bonded to a nucleotide comprising a cytosine base, or a nucleotide comprising an adenine base hydrogen bonded to either a nucleotide comprising a uracil base or a thymine base, are base pairs useful according to the invention.

According to a further aspect of the present invention there is provided a compound comprising nucleotide sequences:

5′-P-N¹-N²-G-N³-C-I-3′ and 3′Q-N⁴-N⁵-N⁶-N⁷-N⁸-N⁹-Y-J-5′

wherein

P and Q are any two nucleotides that can form a Watson-Crick base pair,

I and J are any two nucleotides that can form a base pair, preferably a Watson-Crick base pair,

N¹ and N⁴ are not both C,

when N² is A, N⁷ is not G,

N⁹ can only be U if N³ is A,

when N³ is G, N⁹ is A,

the sequences can anneal to each other, and the compound is not a complete HCV genome or fragment thereof.

As used herein, “anneal” means bind specifically via hydrogen bonding with a complementary nucleotide.

Preferably the sequences of the compounds of the invention bind to each other with a K_{d} of between 1×10⁻¹² and 1×10⁻⁴ M, preferably between 1×10⁻¹² and 1×10⁻⁶ M, more preferably 1×10^{31 12} and 1×10⁻⁸ M and most preferably 1×10⁻¹² and 1×10⁻⁹ M.

As used herein, a compound that is “not a complete HCV genome or a fragment thereof” does not comprise any of the pairs of 5′-P-N¹-N²-G-N³-C-I-3′ and 3′-Q-N⁴-N⁵-N⁶-N⁷-N⁸-N⁹-Y-J-5′ sequences shown in FIG. 3.

The compounds of the present invention comprise the mIRES of an HCV genome or comprise a structure which mimics the mIRES of an HCV genome. Preferably the compounds of the present invention comprise a structure which adopts a conformation substantially identical to a mIRES of an HCV genome and which exhibits a ligand binding pattern substantially identical to a mIRES of an HCV genome.

As used herein, a conformation that is “substantially identical” to that of a mIRES of an HCV genome can support or, preferably, is optimal for, the functions of the wild type mIRES of an HCV genome. The “functions” of the wild type mIRES include, but are not limited to, ligand binding, for example eIF3 binding.

A compound of the invention that exhibits a ligand binding pattern that is “substantially identical” to that of an mIRES of an HCV genome, can support binding of, and, preferably is optimal for binding of, any ligand, as defined herein, that is capable of binding to a mIRES of an HCV genome under suitable conditions.

“Suitable conditions” refers to, for example, an appropriate buffer composition and temperature that supports and is, preferably, optimal for binding. Conditions suitable for binding of an mIRES to eIF3 are described in Example 3, below.

As used herein, a structure which “mimics” the mIRES of an HCV genome exhibits a ligand binding pattern that is substantially identical to the ligand binding pattern of the mIRES of an HCV genome. For example, a structure which “mimics” the mIRES of an HCV genome can bind specifically to eIF3 such that the activity of eIF3 is supported and is preferably optimal. A structure which “mimics” the mIRES of an HCV genome, according to the invention, has the generic formula and structure of a mIRES of an HCV genome as shown in FIG. 2.

Standard nucleotide abbreviations are used herein: A is a nucleotide comprising an adenine base; G is a nucleotide comprising a guanine base; C is a nucleotide comprising a cytosine base; T is a nucleotide comprising a thymine base; U is a nucleotide comprising a uracil base; R is a nucleotide comprising a purine base (i.e. A or G); Y is a nucleotide comprising a pyrimidine base (i.e. C or U or T); and N is any nucleotide. Each occurrence of R, Y or N in a sequence may be the same or different. The term “W.C” refers to a Watson-Crick base pair (i.e. G.C, C.G, A.U, U.A, A.T, T.A).

As used herein the term nucleotide may refer to a deoxyribonucleotide or a ribonucleotide. The compounds of the invention may comprise RNA or DNA, or a mixture of both. Preferably, the compounds of the present invention are RNA.

In the compounds of the invention:

flanking base pairs P.Q and I.J may be any Watson-Crick base pair. P.Q is preferably a R.Y base pair, particularly G.C. I.J is preferably a Y.R base pair, particularly C.G.

N¹ is preferably A or G. When N¹ is A, N⁴ is preferably U or, more preferably, G or A. When N¹ is G, N⁴ is preferably C or, more preferably, A or U.

N² is preferably A, C, G or U. When N² is A, N⁷ is preferably U. When N² is C, N⁷ is preferably C or U or, most preferably, A. When N² is G, N⁷ is preferably C or U. When N² is U, N⁷ is preferably U.

N³ and N⁹ can preferably form an A.U base pair i.e. when N³ is A, N⁹ is preferably U, and when N³is U, N⁹ is preferably A.

N⁴ is preferably A, C, G or U. When N⁴ is A, N² is preferably A or G. When N⁴ is C, N² is preferably G. When N⁴ is G, N² is preferably A. When N⁴ is U, N² is preferably A or G.

N⁵ is preferably U.

N⁶ is preferably G, U or, most preferably, A.

N⁷ is preferably A, C, G or U. When N⁷ is A, N² is preferably C. When N⁷ is C, N² is preferably C or G. When N⁷ is G, N² is preferably U. When N⁷is U, N² is preferably C, A or G.

N⁸ is preferably C.

Y is preferably C.

The compounds of the present invention may comprise a single molecule, for example a single stranded RNA molecule comprising both of the sequences 5′-P-N¹-N²-G-N³-C-I-3′ and 3′-Q-N⁴-N⁵-N⁶-N⁷-N⁸-N⁹-Y-J-5′. Alternatively, the compounds of the present invention may comprise two or more molecules. Preferably, the compounds of the present invention comprise two, annealed molecules, for example two single stranded RNA molecules each, independently, comprising one of the sequences 5′-P-N¹-N²-G-N³-C-I-3′ and 3′-Q-N⁴-N⁵-N⁶-N⁷-N⁸-N⁹-Y-J-5′, wherein the sequences 5′-P-N¹-N²-G-N³-C-I-3′ and 3′-Q-N⁴-N⁵-N⁶-N⁷-N⁸-N⁹-Y-J-5′ are annealed to one another.

Where the compounds comprise a single molecule, the 3′ end of the sequence 5′-P-N¹-N²-G-N³-C-I-3′ and the 5′ end of the sequence 3′-Q-N⁴-N⁵-N⁶-N⁷-N⁸-N⁹-Y-J-5′ joined by a linker which allows the sequences to anneal to each other. Preferably, the compound comprises the sequence 5′-(N)ₐ-[P-N¹-N²-G-N³-C-I]-(N)_{b}-[J-Y-N⁹-N⁸-N⁷-N⁶-N⁵-N⁴-Q]-(N)_{c}-3′ wherein N may or may not be identical and is any nucleotide, a is zero or an integer from 1 to 100 (preferably ≧4, e.g., 5, 10, 20, 30, 50, 80 or 100), b is an integer from 3 to 100 (preferably ≧4, e.g., 5, 10, 20, 30, 50, 80 or 100), and c is zero or an integer from 1 to 100 (preferably ≧4, e.g., 5, 10, 20, 30, 50, 80 or 100). Preferably, a=c.

Preferably, the linker —(N)_{b}— comprises a sequence which can serve to stabilise the mIRES i.e. which maintains the mIRES conformation so that its structure (e.g. secondary and tertiary) is substantially identical, as defined herein, to that of the wild-type mIRES sequence. As used, “wild-type” refers to the normal, non-mutant form of an mIRES sequence. “Wild-type” also refers to an mIRES sequence that normally occurs in nature. The mIRES may, for example, be stabilised by a nucleotide sequence capable of forming a duplex comprising Watson-Crick base pairs, a cross-linked sequence, and/or a sequence capable of forming a secondary structure such as a loop. A “cross-linked sequence” according to the invention can be formed by using a cross linking agent including but not limited to acridine, thio derivatives, aldehydes, azido, bromo groups or ellipticenes (photolytic cross-linking), according to methods well-known in the art.

For example, the linker —(N)_{b}— may comprise nucleotides (RNA or DNA or both) capable of forming a duplex comprising one or more Watson-Crick base pairs adjacent to the mIRES, i.e. adjoining the 3′ end of the sequence 5′-P-N¹-N²-G-N³-C-I-3′and the 5′ end of the sequence 3′-Q-N⁴-N⁵-N⁶-N⁷-N⁸-N⁹-Y-J-5′. Preferably, the linker —(N)_{b}— comprises nucleotides capable of interacting to form from one to ten, preferably two to four, more preferably three, Watson-Crick base pairs adjacent to the mIRES. The linker —(N)_{b}— may also be capable of forming a loop structure. The linker —(N)_{b}— may, for example, comprise the structure:

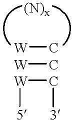

wherein x is an integer from 4 to 20.

Where the compound of the present invention comprises two or more annealed sequences, the compound may comprise a first nucleic acid strand comprising the sequence 5′-P-N¹-N²-G-N³-C-I-3′ annealed to a second nucleic acid strand comprising the sequence 3′-Q-N⁴-N⁵-N⁶-N⁷-N⁸-N⁹-Y-J-5′. Preferably, the compound comprises a first nucleic acid strand comprising the sequence 5′(N)ₐ-P-N¹-N²-G-N³-C-I-(N)_{b}_{¹}-3′ annealed to a second nucleic acid strand comprising the sequence 3′-(N)_{c}-Q-N⁴-N⁵-N⁶-N⁷-N⁸-N⁹-Y-J-(N)_{b}_{²}-5′, wherein a is zero or an integer from 1 to 100, b¹ is zero or an integer from 1 to 100, b² is zero or an integer from 1 to 100, c is zero or an integer from 1 to 100. Preferably, a=c and/or b¹=b² (e.g. FIG. 4).

Preferably, —(N)_{b1}— and —(N)_{b2}— are capable of interacting to stabilise the mIRES. The mIRES may, for example, be stabilised by nucleotides capable of forming a duplex comprising Watson-Crick base pairs, cross-linked sequences and/or a sequence capable of forming a secondary structure such as a loop. Preferably, —(N)_{b1}— and —(N)_{b2}— comprise nucleotide sequences capable of annealing to each other. For example, —(N)_{b1}— and —(N)_{b2}— may comprise nucleotides (RNA or DNA or both) capable of forming a duplex comprising one or more Watson-Crick base pairs adjacent to the mIRES. Preferably, —(N)_{b1}— and —(N)_{b2}— comprise nucleotides capable of forming from one to ten, preferably two to four, more preferably three, Watson-Crick base pairs adjacent to the mIRES.

Preferably, —(N)ₐ— and —(N)_{c}— are capable of stabilising the mIRES. The mIRES may, for example, be stabilised by nucleotides capable of forming a duplex comprising Watson-Crick base pairs, cross-linked sequences and/or a sequence capable of forming a secondary structure such as a loop. Preferably, —(N)ₐ— and —(N)_{c}— comprise sequences capable of annealing to each other. For example, —(N)ₐ— and —(N)_{c}— may comprise nucleotides (RNA or DNA or both) capable of forming a duplex comprising one or more Watson-Crick base pairs adjacent to the mIRES i.e. adjoining the 5′ end of the sequence 5′-P-N¹-N²-G-N³-C-I-3′ and the 3 end of the sequence 3′-Q-N⁴-N⁵-N⁶-N⁷-N⁸-N⁹-Y-J-5′.

Preferably, —(N)ₐ— and —(N)_{c}— are capable of interacting to form from one to forty, preferably one to twenty, Watson-Crick base pairs adjacent to the mIRES. —(N)ₐ— and/or —(N)_{c}— may independently form a loop structure and/or may interact to form a junction structure, such as a four-way junction.

Where the compound is used in an assay based on binding to a reporter molecule, as defined herein below, it is generally desirable to minimise the size of the compound in order to simplify preparation, purification and handling. In such circumstances the compound of the present invention preferably comprises 100 or fewer, more preferably 50 or fewer, more preferably 30 or fewer nucleotides. The ability to use a compound comprising a shortened mimic of the 5′-UTR allows simple and rapid chemical synthesis of the compound, and also aids enzymatic synthesis by, for instance, T7 polymerase. The introduction of chemical modifications is also easier.

It has been found, however, that whilst the mIRES is essential for binding of eIF3, the mIRES alone is not sufficient for binding eIF3. Accordingly, in assays based on binding of the compound to eIF3, the compound of the present invention preferably comprises 50 or more, more preferably 100 or more, and most preferably 150 or more nucleotides.

The compound of the invention preferably binds eIF3 with an affinity ≧2% of the affinity of the interaction between HCV 5′-UTR domain III (DIII) (see, FIG. 5) and eIF3. More preferably, the affinity is ≧10%, and most preferably ≧50% of the affinity of the DIII/eIF3 interaction.

In a preferred embodiment particularly suitable for use in an assay based on binding of the compounds of the invention to eIF3, —(N)ₐ— and —(N)_{c}— are capable of forming a structure comprising:

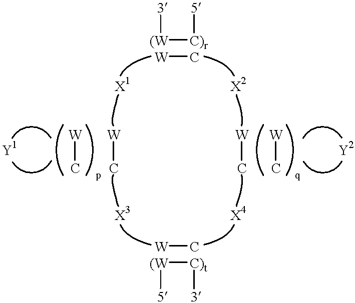

wherein each W-C may or may not be identical and is a Watson-Crick base pair, p1 X¹, X², X³ and X⁴ may or may not be identical and each, independently, comprise from zero to four nucleotides,

Y¹ and Y² may or may not be identical and each, independently, comprise from three to ten nucleotides,

p, q, r and t may or may not be identical and each, independently, comprise integers from zero to ten.

Preferably, —(N)ₐ— and —(N)_{c}— are capable of forming a structure comprising:

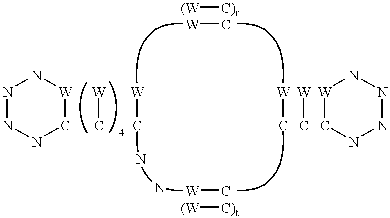

The sequences of the 5′-P-N¹-N²-G-N³-C-I-3′ and 3′-Q-N⁴-N⁵-N⁶-N⁷-N⁸-N⁹-Y-J5′ regions from published HCV sequences are shown aligned in FIG. 3. It will be noted, for instance, that the 5′ residue of the sequence of FIG. 3A has been selected by nature to be G or, in a single case, A. The complementary residue of the sequences of FIG. 3B is similarly, therefore, either C or, in a single case, U. According to the invention, however, there is no such restriction.

The invention provides for sequences wherein P and Q are any two nucleotides that can form a Watson-Crick base pair.

The invention also provides for sequences wherein I and J are any two nucleotides that can form a base pair, preferably a Watson-Crick base pair.

Thus, in preferred compounds of the invention: P is a purine; Q is a pyrimidine; I is a pyrimidine; J is a purine; N¹ is A, G or U; N² is A, C or U; N³ is A; N⁴ is a purine; N⁵ is U; N⁶ is a purine; N⁷ is A, G or U; N⁸ is C; N⁹ is U; and Y is C. These compounds represent the sequences of FIG. 3, which is an alignment of the sequences from published HCV genomes.

The sequences 5′-P-N¹-N²-G-N³-C-I-3′ and 3′-Q-N⁴-N⁵-N⁶-N⁷-N⁸-N⁹-Y-J-5′ employed in the present invention may comprise sequences corresponding to wild-type HCV sequences or may comprise sequences which do not consist of naturally occurring HCV sequences.

Where the sequences do consist of two wild-type HCV sequences, the sequences may be one of the 35 pairs of sequences shown in FIG. 3. Preferably, the sequences are 5′-G-A-C-G-A-C-C-3′ and 3′-C-G-U-A-A-C-U-C-G-5′, the sequences being from the prototype genome of strain 1a (M67463).

Further features and advantages of the invention will become more fully apparent in the following description of the embodiments and drawings thereof, and from the claims.

### BRIEF DESCRIPTION OF DRAWINGS

FIGS. 1A and B shows the 5′-UTR of HCV strain Ia (M67463). The boxed region is the mIRES of the invention.

FIG. 2 shows the mIRES in generalised structural formula.

FIG. 3 is an alignment of the two short sequences that make up the mIRES from published HCV genomic sequences (shown in DNA form, with T instead of U). These 35 pairs of sequences are those which were represented more than once in an alignment of more than 600 wild-type HCV sequences.

FIG. 4 illustrates the mIRES of FIG. 1 in a heterologous context, flanked by at least five Watson-Crick base pairs on each side.

FIG. 5 shows HCV 5′-UTR Domain III EMSA RNA probes.

FIG. 6 shows (a) a fluorescein labelled RNA construct containing the mIRES and (b) a 19-mer RNA control construct.

FIG. 7 shows binding cures for the RNA construct containing the mIRES titrated with various peptides.

FIG. 8a shows the interaction between paromomycin-TAMRA and IIIB-Dabcyl RNA

FIG. 8b shows inhibition of paromomycin-TAMRA and IIIB-Dabcyl RNA binding by neomycin.

### DESCRIPTION

The invention provides for compounds comprising nucleotide sequences 5′-P-N¹-N²-G-N³-C-I-3′ and 3′-Q-N⁴-N⁵-N⁶-N⁷-N⁸-N⁹-Y-J-5′ that are the generic sequences of a mIRES of an HCV genome. The invention also provides assays that utilise the compounds of the invention, including assays for detection of molecules that interact with the HCV genome, including, HCV antiviral molecules.

As defined herein the invention encompasses

1. Shortened 5′-UTR—The mIRES is much smaller than the complete 5′-UTR. Therefore in one aspect the invention provides a compound comprising sequences 5′-P-N¹-N²-G-N³-C-I-3′ and 3′-Q-N⁴-N⁵-N⁶-N⁷-N⁸-N⁹-Y-J-5′, wherein said sequences are capable of annealing to each other, characterised in that the molecule comprises 200 or fewer nucleotides.

2. Heterologous context—The mIRES is able to bind eIF3 and other reporter molecules in contexts other than the native 5′-UTR. The invention therefore provides the mIRES in a heterologous context, that is to say, flanked on one or both sides by sequences not found in an HCV genome. The ability to place the mIRES in a heterologous context, for instance being flanked by stem structures, allows a very stable molecule to be produced. Furthermore, only nine consecutive nucleotides from the wild-type HCV sequence are required for function (i.e., functional eIF3 binding).

3. Double-stranded—The wild-type HCV genome consists of a single RNA strand. Stem IIIb and the mIRES are formed from this single strand folding back on itself to form a duplex region. The identification of the mIRES allows the construction of a functional molecule from two separate nucleic acid strands. Accordingly, in this aspect, the invention provides a compound comprising a first nucleic acid strand comprising the sequence 5′-P-N¹-N²-G-N³-C-I-3′ annealed to a second nucleic acid strand comprising the sequence and 3′-Q-N⁴-N⁵-N⁶-N⁷-N⁸-N⁹-Y-J-5′. The ability to form the mIRES from separate strands simplifies the chemical synthesis of the compounds of the invention. It also facilitates differential labelling of the two strands.

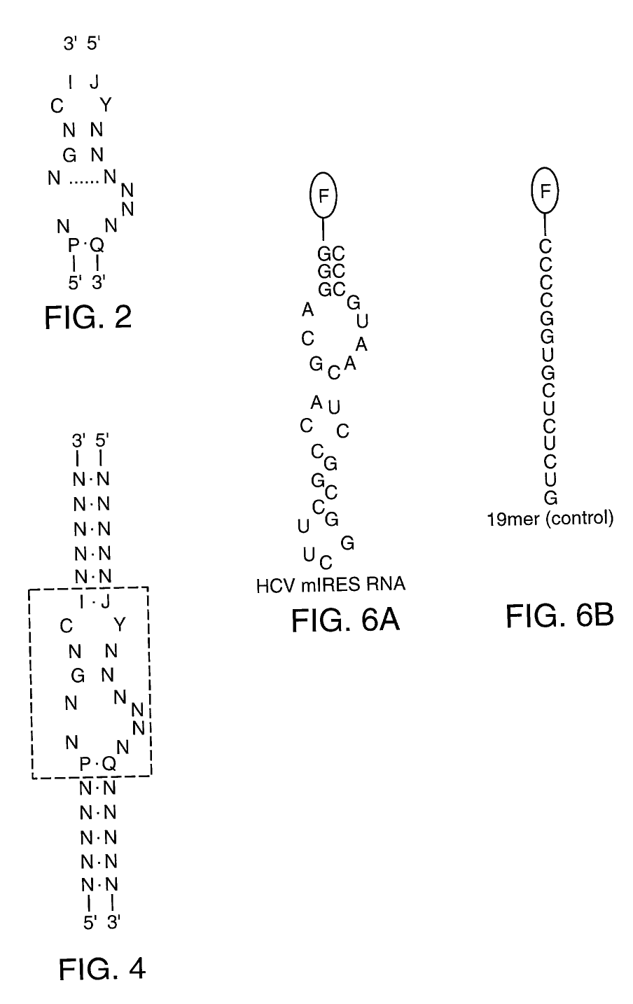

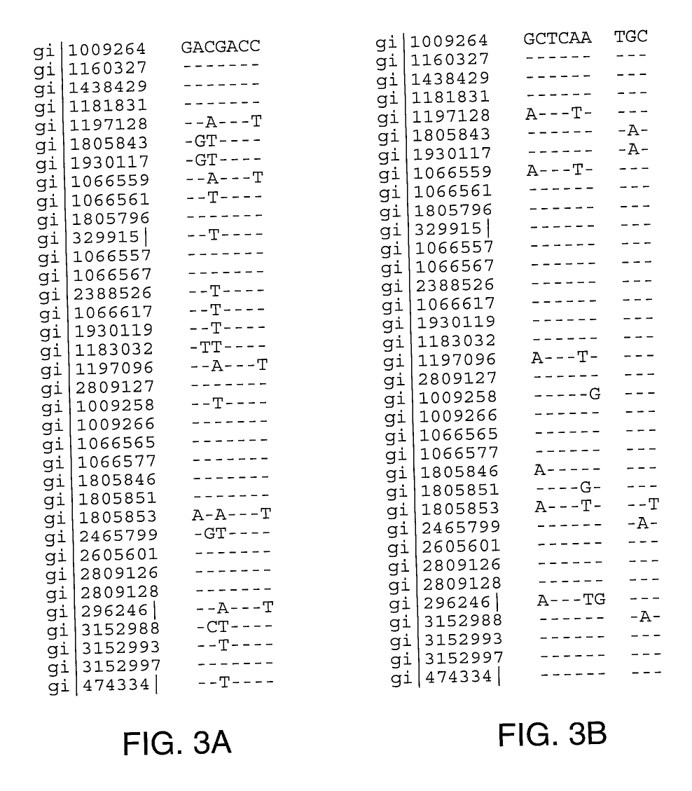

4. Modified mIRES—As well as identifying a minimal fragment of the 5′-UTR that is essential for binding eIF3, the mIRES has been dissected internally to determine its critical residues. The generic formula of the mIRES that is the subject of the present invention is shown in FIG. 2. Knowledge of this generic formula permits mutation of the mIRES whilst retaining functional binding, for example to eIF3. This allows desired sequences and/or non-wild-type sequences to be incorporated into the mIRES without losing function. Modified mIRES can be presented in the same manner as described above for the native mIRES (i.e. in shortened, heterologous and/or double stranded context). In this aspect of the invention, where the compound is not a complete HCV genome or a fragment thereof, the sequences 5′-P-N¹-N²-G-N³-C-I-3′ and 3′-Q-N⁴-N⁵-N⁶-N⁷-N⁸-N⁹-Y-J-5′ should not be one of the pairs shown in FIG. 3.

### Production

The molecules of the invention may be produced in various ways, including transcription from DNA templates by RNA polymerase, by enzymatic replication of RNA templates, or by chemical synthesis with an automated oligonucleotide synthesiser, according to methods well known in the art. Suitable RNA polymerases for transcription include the bacterial polymerases T3 and T7, Sp6, and E.coli polymerase. Suitable RNA replicases include the replicase from Qβ RNA polymerase.

### Modifications

RNA is sensitive to cleavage by cellular ribonucleases, as well as to alkaline or acid conditions. The molecules of the invention may therefore contain modifications that confer greater stability. Modifications may also be desirable to provide groups for immobilising the RNA on solid supports by covalent or non-covalent attachments. Typical modifications include modified bases and/or sugars and/or linkages. The terms “RNA molecule”, “nucleotide” and “oligonucleotide” as used herein are intended to cover all such variants.

Modifications may include, but are not limited to, the following types:

a) Backbone modifications:

(i) phosphorothioates (single S substituents or any combination of two or more with remainder as O,

(ii) methylphosphonates

(iii) phosphoramidates

(iv) phosphotriesters

(v) phosphorus-free linkages (e.g. carbamate, acetamidate, acetate,)

b) Sugar modifications:

(i) 2′-deoxynucleosides (R═H)

(ii) 2′-O-methylated nucleosides (R═OMe)

(iii) 2′-fluoro-2′-deoxynucleosides (R═F)

(iv) 2′-O-alkylated nucleosides

c) Base modifications:

(i) pyrimidine derivatives substituted in the 5-position (e.g. methyl, bromo, fluoro etc. or replacing a carboxyl group by an amino group,

(ii) purine derivatives lacking specific nitrogen atoms (e.g. 7-deaza-adenine, hypoxanthine, or functionalised in the 8-position (e.g. 8-azido adenine, 8-bromo adenine), or additional functionalities (.e.g. 2,6-diaminopurine).

d) Oligonucleotides covalently linked to reactive functional groups (e.g. psoralens, phenanthrolines, mustards).

e) irreversible cross-linking agents with or without the need for co-reagents)

(i) acridine (intercalating agents)

(ii) thio derivatives (reversible disulphide formation with proteins)

(iii) aldehydes (Schiff's base formation)

(iv) azido, bromo groups (UV cross-linking)

(v) ellipticenes (photolytic cross-linking)

f) oligonucleotides containing haptens or other binding groups;

g) fluorescent moieties or other non-radioactive labels; and

h) combinations of two or more modifications selected from (a) to (g) above.

### Assays

The above-defined compounds of the invention can be used in a variety of ways. Of particular interest is their use in assays, particularly screening assays. Rather than screen potential antivirals (test compounds or test molecules) against virus in culture, or against artificial reporter gene constructs, they can be screened against a compound of the invention. Compounds that interact with the mIRES will typically inhibit the formation of the eIF3/5′-UTR complex in vivo.

Therefore the invention provides a screening assay, comprising the steps of (a) incubating a test molecule with a compound of the invention, and (b) detecting the formation of a binding complex.

In one embodiment, a “binding complex” according to the invention comprises a test molecule bound specifically to a compound of the invention. In another embodiment, a “binding complex” comprises a compound according to the invention bound specifically to a ligand as defined hereinbelow. In another embodiment, a “binding complex” according to the invention comprises a pair of indicator molecules bound specifically to each other. In this aspect of the invention, the pair of indicator molecules can be a ligand comprising a labelled reporter, and a labelled compound according to the invention. In one aspect of this embodiment, each member of the pair of indicator molecules is independently labelled with a complementary acceptor and donor group.

As used herein, “bound specifically” or “bind specifically” means associate(d) by hydrogen bonding, covalent bonding, electrostatic interaction or via an interaction between, for example, a molecule (e.g., a polypeptide or a nucleic acid) and a ligand, as defined herein, protein subunits, a nucleic acid binding protein and a nucleic acid binding site or between complementary nucleic acids, as a result of attractive forces that exist between the members of a binding complex according to the invention.

Methods of detecting the formation of a binding complex or determining the amount of a binding complex are described in the section entitled, “Measurable Changes”.

The assay may involve a test molecule or compound of the present invention which is labelled (e.g. with an isotopic or non-isotopic label, such as a fluorescent label). The binding complex can be detected by monitoring changes in the signal of the label when the test molecule binds to the compound of the present invention. As used herein, a “change” in a signal can be an increase or a decrease in the amount or intensity of a signal, for example an increase or a decrease in fluorescence. In one embodiment, a “change” in a signal means a different emission spectra, for example as may occur if FRET between a donor and an acceptor gives rise to a binding complex with a distinct fluorescence emission spectra as compared to the fluorescence emission spectra of the individual members of the binding complex, in an uncomplexed form. As an alternative, the assay may involve the compound of the invention immobilised on a solid surface and a labelled test molecule, with the binding complex being detected by detecting label bound to the solid surface.

According to one embodiment of the present invention there is provided a screening assay comprising the steps of (a) incubating a test molecule with a compound of the present invention and with a ligand capable of binding to the compound of the present invention, and (b) determining the amount of binding complex comprising the compound of the present invention and the ligand.

The ligand and the compound of the present invention may be separate or may be in the form of a complex before addition of the test molecule. The assay may measure the extent to which the test molecule inhibits the formation of a binding complex comprising the compound of the present invention and the ligand, or may measure the extent to which the test molecule displaces the ligand from complexation with the compounds of the present invention.

Ligands useful in the present invention are molecules capable of binding specifically to a compound of the present invention and may be readily identified as described herein. Preferably, the ligand is a polypeptide. In one embodiment, the ligand may comprise eIF3 or a fragment thereof. Fragments of eIF3 include the individual polypeptide chains which make up eIF3, which include p170, p116, p110, p66, p48, p47, p44, p40, p37, and p35 [ref. viii, page 3186]. Thus in one embodiment, the invention provides a screening assay, comprising the steps of (a) incubating a potential HCV antiviral molecule with a compound of the invention and eIF3 or a fragment thereof, and (b) determining the amount of binding complex comprising the compound of the invention and eIF3 or a fragment thereof.

As used herein, an “HCV antiviral molecule” binds specifically to a compound of the invention. In one embodiment, an “HCV antiviral molecule” binds specifically to a compound of the invention and inhibits the formation of a binding complex comprising a compound of the invention, and a ligand, as defined herein. For example, an “HCV antiviral molecule” can inhibit the formation of an eIF3/5′ UTR complex. In another embodiment, an “HCV antiviral molecule” binds specifically to a compound of the invention and inhibits the formation of a binding complex comprising a pair of indicator molecules, for example, a labelled compound of the invention and a ligand reporter, as defined herein. In another embodiment, an “HCV antiviral molecule” binds specifically to a compound of the invention and inhibits the formation of a binding complex comprising a target, as defined herein, and a ligand, as defined herein. As used herein, “inhibits the formation of a binding complex” means decreases the amount of binding complex, as compared to the amount of binding complex in the absence of an HCV antiviral molecule, by at least 5%, preferably 10-50% and most preferably, 50-100%.

Preferably the ligand is capable of binding the mIRES.

In one embodiment the assay may be based on that described in PCT/GB99/01761, which is incorporated herein by reference. The assay employs a target molecule comprising a compound of the present invention; and a reporter molecule comprising a ligand capable of binding to the compound of the present invention.

According to this aspect, the invention provides a method for determining whether a test molecule can bind specifically to an HCV 5′-UTR, the method comprising the steps of:

(a) contacting the test molecule with a pair of indicator molecules comprising (i) a reporter labelled with a donor group or an acceptor group and (ii) a compound according to the present invention labelled with a complementary acceptor or donor group, the pair being capable of binding specifically to each other in an orientation that permits the donor group to come into sufficient proximity to the acceptor group to permit fluorescent resonance energy transfer and/or quenching to take place; and

(b) measuring the fluorescence of the compound of the present invention and/or the reporter ligand in the presence of the test molecule and comparing this value to the fluorescence of a standard.

In preferred embodiments, the standard comprises:

the indicator pair in the absence of test molecule;

the indicator pair in the presence of test molecule;

individual member(i) of the indicator pair in the absence of test molecule;

individual member (ii) of the indicator pair in the absence of test molecule;

individual member (i) of the indicator pair in the presence of test molecule; and/or

individual member (ii) of the indicator pair in the presence of test molecule.

It will be appreciated that various combinations of these six choices can be used.

It will be appreciated that the fluorescence of the standard may have been determined before performing the method, or may be determined during or after the method has been performed. It may be an absolute standard.

In relation to this aspect of the present invention, the compounds of the present invention are referred to as the target.

As used herein, “contacting” refers to combining or mixing, in any order, and preferably mixing, as described in the section entitled, “order of mixing”.

The method may also be used in the identification of molecules that bind to the target from within a plurality of test molecules, such as in screening methods. The method may, therefore, involve the initial step of providing a plurality of test molecules.

In a typical embodiment, therefore, the invention provides a method of screening for potential antiviral molecules, comprising the steps of (a) contacting a test molecule with an indicator complex, the indicator complex comprising a fluorescently-labelled reporter ligand bound to a fluorescently labelled compound of the present invention in an orientation that permits the fluorescent groups present on each molecule to come into sufficient proximity to permit fluorescent resonance energy transfer to take place; and (b) measuring the fluorescence of the compound of the present invention and the reporter ligand in the presence of the test molecule and comparing this value to the fluorescence of a standard.

### The Reporter Ligand

The reporter ligand is capable of binding to the target and preferably forms a one-to-one complex with the target. Reporter ligands can thus be virtually any agent including, without limitation, peptides, peptoids, proteins, lipids, polysaccharides, derivatives of these, and small organic molecules with molecular weights of more than 200 and less than about 2,500 daltons, preferably between 500 and 1,000 daltons.

The reporter preferably binds the target with a K_{d} of between 1×10⁻¹² and 1×10−4 M. Preferred values are lower than 500 nM.

In preferred embodiments of the invention, the reporter ligand comprises a linear peptide or derivative thereof, a cyclic peptide or derivative thereof, a linear or cyclic peptoid or derivative thereof, or a peptidomimetic analogue. Linear peptides, peptoids and derivatives thereof are between 2 and 100 residues in length, preferably between 4 and 40 residues in length, and most preferably between 8 and 20 residues in length and may comprise either D- or L-amino acids (or equivalents). Cyclic peptides, peptoids and derivatives thereof are preferably between 4 and 10 residues in length, preferably 4 to 7, and may comprise either D- or L-amino acids (or equivalents). Peptoids are isomers of peptides which have side chains carried by backbone nitrogens (N-substituted glycines) (eg. Bartlett, et al. WO91/19735, Zuckermann, et al. WO94/0645 1 and Simon et al., 1992). Peptoids are more flexible than peptides since intramolecular CO—HN hydrogen bonds are removed and the steric interactions that induce secondary structure are different.

### Fluorescent Labelling

The target and the reporter ligand may be fluorescently labelled by any suitable method, preferably by covalent attachment of a fluorescent group. The labels may be any fluorescent label or fluorophore that does not interfere with the ability of the reporter to interact with the target and is able to show quenching and/or fluorescence resonance energy transfer with the corresponding label on the target.

The target may be fluorescently labelled at any suitable position. In some embodiments, the fluorescent group or quenching group is placed on or adjacent to the 5′ end of the RNA target. In other embodiments, it may be placed on or adjacent to the 5′ end of one of a pair of oligonucleotides forming an RNA duplex, or the 5′ end of one of the component oligonucleotides in an RNA structure created by the annealing of three or more RNA oligonucleotides. In other embodiments, the fluorescent group may be placed on or adjacent to the 3′ end of one of the synthetic RNA molecules.

In other embodiments, the fluorescent group may be placed within the chain of the synthetic RNA molecules, for instance by incorporation of a fluorescent nucleotide derivative, modification of a nucleotide or substitution of a nucleotide by a fluorescent molecule. For example, tetramethylrhodamine (TAMRA) can be introduced into synthetic RNA by incorporating the modified deoxy-uridine phosphoramidite (5′-Dimethoxytrityloxy-5-[N-((tetramethylrhodaminyl)-aminohexyl)-3-acryimido]-2′-deoxy-uridine-3′-[(2-cyanoethyl)-(N,N-diisopropyl)]-phosphoramidite). Fluorescein may be incorporated in an analogous way with: 5′-Dimethoxytrityloxy-5-[N-((3′,6′-dipivaloylfluoresceinyl)-aminohexyl)-3-acryimido]-2′-deoxy-uridine-3′-[(2-cyanoethyl)-(N,N-diisopropyl)]-phosphoramidite. The DABCYL group may also be incorporated using 5′-Dimethoxytrityloxy-5-[N-((4-(dimethylamino)azobenzene)-aminohexyl)-3-acryimido]-2′-deoxy-uridine-3′-[(2-cyanoethyl)-(N,N-diisopropyl)]-phosphoramidite. More generally, a free amino group may be reacted with the active ester of any dye, such an amino group may be introduced by the inclusion of the modified uridine 5′-Dimethoxytrityl-5-[N-(trifluoroacetylaminohexyl)-3-acrylimido]-2′-deoxy-uridine,3′-[(2-cyanoethyl)-(N,N-diisopropyl)]-phosphoramidite. The incorporation of a single deoxy-uridine often does not significantly perturb RNA structure and the modification at the 5 position of the base allows for normal base-pairing.

It is also possible to include more than one fluorescent label on a synthetic target molecule without departing from the scope of the invention. In one embodiment of this invention, a target molecule is labelled with two fluorescent groups, for example with one group placed adjacent to the 5′ end of the target RNA sequence and a second fluorescent group placed adjacent to the 3′ end of the target RNA sequence. In other embodiments, two or more fluorescent groups are placed adjacent to the 5′ and/or 3′ ends of the target RNA molecule and/or at internal sites in the RNA target sequences. Multiply labelled target RNAs can be used to increase the intensity of the signals detected in the assay.

In certain embodiments of the invention a target labelled at two or more positions may be used to detect interactions with two or more reporter ligands. The reporter ligands can either be used individually or simultaneously.

The reporter ligand may also be labelled at any suitable position. When peptides or peptoids are used as reporter ligands, the fluorescent group may, for instance, be placed at either the carboxyl or amino terminus of the ligand. In other embodiments using peptides or peptoids the fluorescent group may be placed on a side chain within the peptide or peptoid sequence.

It is also possible to include more than one fluorescent label on the reporter ligand without departing from the scope the invention. Multiply labelled reporter ligand can be used to enhance signal intensity and/or selectivity.

Useful fluorophores (in addition to those listed in PCT/GB99/01761) include, but are not limited to: Texas Red™ (TR), Lissamine™ rhodamine B, Oregon Green™ 488 (2′,7′-difluorofluorescein), carboxyrhodol and carboxyrhodamine, Oregon Green™ 500, 6-JOE (6-carboxy-4′,5′-dichloro-2′,7′-dimethyoxyfluorescein), eosin F₃S (6-carboxymethylthio-2′,4′,5′,7′-tetrabromo-4,5,7-trifluorofluorescein), Cascade Blue™ (CB), aminomethylcoumarin (AMC), pyrenes, dansyl chloride (5-dimethylaminonaphthalene-1-sulfonyl chloride) and other napththalenes, PyMPO, ITC (1-(3-isothiocyanatophenyl)-4-(5-(4-methoxyphenyl)oxazol-2-yl)pyridinium bromide).

### Donor/acceptor Pairing

Contact between the pair of indicator molecules may occur in solution (eg. a test tube, dish or well of a microtitre plate) or, alternatively, either the reporter ligand or the target molecule may be adhered to a solid support (eg. an affinity gel, matrix, or column) by covalent or non-covalent linkages using methods known in the art. The support bound target or reporter molecule is then mixed with a solution containing the other compound of the indicator pair.

When the reporter and target are mixed, they can form a complex which brings the donor and acceptor groups into proximity. The fluorescence of, or light emitted from, the complex formed between the reporter molecule and the target is altered by fluorescence resonance energy transfer (FRET).

The donor group may be attached to either the target or to the reporter ligand. When the donor is attached to the target, the complementary acceptor is attached to the reporter ligand; conversely, when the donor is attached to the reporter ligand, the complementary acceptor is attached to the target.

As used herein, the term “donor” refers to a fluorophore which absorbs at a first wavelength and emits at a second, longer wavelength. The term “acceptor” refers to a fluorophore, chromophore or quencher with an absorption spectrum which overlaps the donor's emission spectrum and is able to absorb some or most of the emitted energy from the donor when it is near the donor group (typically between 1-100 nm). If the acceptor is a fluorophore capable of exhibiting FRET, it then re-emits at a third, still longer wavelength; if it is a chromophore or quencher, then it releases the energy absorbed from the donor without emitting a photon.

Although the acceptor's absorption spectrum overlaps the donor's emission spectrum when the two groups are in proximity, this need not be the case for the spectra of the molecules when free in solution. Acceptors thus include fluorophores, chromophores or quenchers that, following attachment to either the target molecule or to the reporter ligand, show alterations in absorption spectrum which permit the group to exhibit either FRET or quenching when placed in proximity to the donor through the binding interactions of two molecules.

The donor and acceptor groups may independently be selected from suitable fluorescent groups, chromophores and quenching groups. Preferred donors and acceptors include:

5-FAM (also called 5-carboxyfluorescein; also called Spiro(isobenzofuran-1(3H), 9′-(9H)xanthene)-5-carboxylic acid,3′,6′-dihydroxy-3-oxo-6-carboxyfluorescein);

5-Hexachloro-Fluorescein ([4,7,2′,4′,5′,7′-hexachloro-(3′,6′-dipivaloylfluoresceinyl)-6-carboxylic acid]);

6-Hexachloro-Fluorescein ([4,7,2′,4′,5′,7′-hexachloro-(3′,6′-dipivaloylfluoresceinyl)-5-carboxylic acid]);

5 -Tetrachloro-Fluorescein ([4,7,2′,7′-tetrachloro-(3′,6′-dipivaloylfluoresceinyl)-5-carboxylic acid]);

6-Tetrachloro-Fluorescein ([4,7,2′,7′-tetrachloro-(3′,6′-dipivaloylfluoresceinyl)-6-carboxylic acid]);

5-TAMRA (5-carboxytetramethylrhodamine; Xanthylium, 9-(2,4-dicarboxyphenyl)-3,6-bis(dimethylamino);

6-TAMRA (6-carboxytetramethylrhodamine; Xanthylium, 9-(2,5-dicarboxyphenyl)-3,6-bis(dimethylamino);

EDANS (5-((2-aminoethyl)amino)naphthalene-1-sulfonic acid);

1,5-IAEDANS (5-((((2-iodoacetyl)amino)ethyl) amino)naphthalene-1-sulfonic acid);

DABCYL (4-((4-(dimethylamino)phenyl) azo)benzoic acid);

Cy5 (Indodicarbocyanine-5);

Cy3 (Indodicarbocyanine-3); and

BODIPY™ FL (4,4-difluoro-5,7-dimethyl-4-bora-3a,4a-diaza-s-indacene-3-propionic acid)

as well as suitable derivatives thereof.

In preferred embodiments, the target molecule has been specifically labelled by a donor/acceptor that is different from the acceptor/donor that is present on the reporter molecule. Preferred combinations of donors and acceptors are listed, but not limited to, the donor/acceptor pairs shown in PCT/GB99/01761.

As used herein, references to “fluorescence” or “fluorescent groups” or “fluorophores” include luminescence, luminescent groups and suitable chromophores, respectively. In the present invention the target and reporter molecule may be labelled with luminescent labels and luminescence resonance energy transfer is indicative of complex formation. Suitable luminescent probes include, but are not limited to, the luminescent ions of europium and terbium introduced as lanthium chelates (Heyduk & Heyduk, 1997). The lanthanide ions are also good donors for energy transfer to fluorescent groups (Selvin 1995). Luminescent groups containing lanthanide ions can be incorporated into nucleic acids utilising an ‘open cage’ chelater phosphoramidite.

In certain embodiments of the invention, the target and reporter molecule may also be labelled with two chromophores, and a change in the absorption spectra of the label pair is used as a detection signal, as an alternative to measuring a change in fluorescence.

### Measurable Changes

In this embodiment of the present invention, the labelled reporter is capable of binding to the labelled target, thereby forming a complex in which the donor present on one molecule comes into proximity with the acceptor on the other molecule. This results in reduced fluorescence of the complex compared to the uncomplexed fluorescence exhibited by the reporter molecule and/or target when free in solution.

In this embodiment of the invention, fluorescence intensity of the reporter molecule, the fluorescence intensity of the target and the fluorescence intensity of the complex is measured at one or more wavelengths with a fluorescence spectrophotometer or microtitre plate reader. It is generally preferred that the reporter molecule and target form a one-to-one complex and equimolar concentrations of reporter molecule and target are present in the binding reaction. However, an excess of one reagent may be used without departing from the scope of the invention.

In some embodiments, a fraction of the reporter molecules and target molecules in the binding reaction can be replaced by unlabelled analogues. The optimal proportions of labelled and unlabelled reporter and target molecules can be determined by titration of the different components and measuring the optimal concentrations required in order to obtain maximal FRET or fluorescent quenching.

The labelled target and labelled reporter molecules are then mixed with a test molecule and the fluorescence in the mixture is measured. If the test molecule is able to bind to the region of the target that binds to the reporter molecule, then a fraction of the reporter molecule will be prevented from binding to the target. The proportions of the free reporter, free target and complex can be quantitatively determined by comparing the spectral properties of the complex, partially dissociated complex and the uncomplexed target and reporter molecules. The amount of reporter displacement will be a function of the relative affinity of the test molecule for the target compared to the reporter molecule and the relative concentrations of the two molecules in the sample. Preferably, a variety of different concentrations of the molecule to-be-tested are compared to generate a binding curve. Saturation of the target is reached when the fluorescence emission of the reporter or target molecule is restored to the levels obtained from the free molecules.

The concentration of test molecules binding to targets can be determined with a fluorescence standard curve depicting the fluorescence of the labelled reporter and target with varying known concentrations of competing unlabelled test molecule.

In some embodiments of the invention, fluorescence resonance energy transfer between the donor and acceptor may give rise to a distinct fluorescence emission spectrum of the complex which can be compared to the fluorescence emission spectra of the separate reporter and target molecules.

In preferred embodiments of the invention, FRET is detected by steady state measurements of the integrated emission intensity of the donor (i.e. the fluorescent dye that is excited by the light source used in the spectral measurement) and/or the acceptor (i.e. the fluorescent dye which has an absorption spectrum that overlaps the emission spectrum of the donor). In other embodiments of the invention FRET may be detected by time-resolved measurements in which the decay of donor fluorescence is measured after a short pulse of excitation. In certain embodiments of the invention the donor is excited at a wavelength that does not itself result in efficient excitation of the acceptor, and FRET is detected by measuring the excitation of the acceptor due to transfer of a photon from the donor.

The transfer of energy from donor to acceptor is associated with a reduction of the intensity of the fluorescence exhibited by the donor (quenching). In certain preferred embodiments of the invention, only quenching of the donor due to the proximity of the acceptor in the reporter/target complex is measured. In certain embodiments of the invention, the target carries a chromophore or fluorophore that quenches the fluorescence of the fluorescent group on the reporter after binding of the two molecules. In other embodiments of the invention the reporter carries a chromophore or fluorophore that quenches the fluorescence of the fluorescent group on the target after binding of the two molecules.

It will typically be preferable to look for a signal (a positive), rather than for the absence of a signal (a negative), but it will be appreciated that either or both may be followed.

### Order of Mixing

In this embodiment of the invention it is preferred that the target, the reporter, and the test molecule are mixed, and the fluorescence of the mixture is compared to standards. Competitive inhibitors of the binding of the reporter molecule prevent the formation of the reporter-target complex and therefore increase the amount of free target and free reporter in the reaction. Since the fluorescence of the free target and reporter molecules is unquenched, the overall fluorescence in the reaction increases in direct relation to the amount of test molecule in the binding reaction and its relative affinity for the target compared to the reporter molecule.

Alternatively, the test molecule is first mixed with the labelled target in order to form a complex in the absence of the labelled reporter, and the reporter is then added. Since the reporter molecule will only be able to bind to the free target in the reaction, there will be a reduced amount of complex formed between the reporter and the target compared to the amount of complex formed in the absence of test molecule. As a result, the fluorescence of the mixture containing the test molecule will be increased compared to a similar mixture prepared in the absence of the test molecule.

In another alternative, a complex is pre-formed between the labelled target and the labelled reporter molecule before addition of the test molecule. If the test molecule is able to disrupt the complex formed between the labelled-target and the labelled-reporter molecule, or alter the equilibrium binding state by binding to target that has dissociated from the reporter molecule, the amount of complex in the reaction will be reduced and the overall fluorescence of the mixture will increase.

In some circumstances the test molecule may itself be fluorescent and/or be capable of quenching the fluorescent group present on the target and/or the reporter molecule. Preferably, the fluorescence of standards containing the test molecule on its own, and in pairwise combinations with the target or reporter molecules, are measured and these values are compared to the fluorescence of the complete test mixture containing the test molecule, the fluorescent target and the reporter molecule.

Quenching of fluorescence arising from the target due to the binding of the test molecule to the target will result in a decrease in the signal arising from the target that is not complexed to the reporter molecule, but will not affect the fluorescent signal arising from the group on the reporter molecule or the signal obtained from the target in a complex with the reporter molecule. In this circumstance it is preferable to configure the donor/acceptor pairs on the target and the reporter molecule such that an increase in the fluorescence of the reporter molecule is detectable when the formation of the complex between the reporter and the target is blocked by the test molecule.

### Library Screening (Including High Throughput Screens)

The present invention also encompasses high-throughput screening methods for identifying molecules or ligands that bind to the target. Preferably, all the biochemical steps for this assay are performed in a single solution in, for instance, a test tube or microtitre plate, and the test molecules or ligands are analysed initially at a single compound concentration. For the purposes of high throughput screening, the experimental conditions are adjusted to achieve a proportion of test molecules or ligands identified as “positive” compounds from amongst the total compounds screened. The assay is preferably set to identify molecules or ligands with an appreciable affinity towards the target eg. when 0.1% to 1% of the total test molecules or ligands from a large compound library are shown to bind to a given target with a Kᵢ of 10 μM or less (eg. 1 μM, 100 nM, 10 nM, or less).

### Reporter Identification

The methods of the invention require a suitable reporter molecule. Accordingly, another aspect of the invention is a method for identifying a reporter molecule from a mixture (eg. a combinatorial library) of labelled peptides, peptoids or other polymers carrying side chains.

In one example of such a method, a series of peptides or peptoids between 3 and 100 residues in length are synthesised with a mixed collection of side chains (containing either natural amino acid side chains or sequence variants) at several positions within the sequence (eg. Felder et al., WO96/40759, and Hamy et al., 1997) and a fluorescent moiety (either a donor or an acceptor) is placed at either the C-terminal or N-terminal end.

The individual reporter candidates are then mixed in solution with the target of interest that has been labelled with a complementary donor or acceptor. Complexes between the individual reporter candidates and the target are detected by measuring quenching and/or fluorescence resonance energy transfer. Preferably, the reporter candidates are tested over a range of different concentrations (between 10 nM and 1 mM) and the target is at a fixed concentration (between 10 nM and 100 nM). The reporter candidates are then ranked by calculating K_{d} for each compound and the target RNA pair. The reporter candidate with the lowest K_{d} is then selected for use as a reporter.

### Test Molecules or Ligands

Test molecules or ligands from large libraries of synthetic or natural compounds can be screened. Numerous means are currently used for random and directed synthesis of saccharide, peptide, and nucleic acid based compounds. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are available and can be prepared. Alternatively, libraries of natural compounds in the form of bacterial, fungal, plant and animal extracts are available from e.g., Pan Laboratories (Bothell, Wash.) or MycoSearch (NC), or are readily produceable by methods well known in the art. Additionally, natural and synthetically produced libraries and compounds are readily modified through conventional chemical, physical, and biochemical means.

Useful compounds (including test molecules and ligands) may be found within numerous chemical classes. Useful compounds (including test molecules and ligands) may be organic compounds, or small organic compounds. Small organic compounds have a molecular weight of more than 50 yet less than about 2,500 daltons, preferably less than about 750, more preferably less than about 350 daltons. Exemplary classes include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

### Kits of the Invention

The invention also provides a kit for determining whether a test molecule binds to a compound of the present invention, the kit comprising (a) a compound of the present invention and (b) a ligand capable of binding the compound of the present invention, wherein either or both the ligand and the compound of the present invention are labelled.

In a preferred embodiment, the invention provides a kit for determining whether a test molecule is capable of binding to an HCV 5′-UTR, the kit comprising (a) a compound of the present invention labelled with a donor group or an acceptor group and (b) a reporter labelled with a complementary acceptor or donor group, wherein the reporter and the compound of the present invention are capable of binding to each other in an orientation that permits the donor group to come into sufficient proximity to the acceptor group to permit fluorescent resonance energy transfer and/or quenching.

In a preferred embodiment, the invention provides a kit for determining whether a test molecule is an HCV antiviral molecule, as defined herein.

The invention also provides an HCV antiviral molecule identified by such a screening assay and use of such an antiviral in the treatment of HCV.

There is also provided a method for formulating an HCV antiviral, comprising the steps of (a) performing a screening assay according to the invention, (b) selecting an antiviral molecule that inhibits the formation of an eIF3/5′-UTR mimic complex, or inhibits the formation of a binding complex comprising a compound of the invention and a ligand, as defined herein, and (c) formulating the antiviral as a pharmaceutical.

### Dosage and Mode of Administration

By way of example, a patient in need of an HCV antiviral compound, for example, identified in a screening assay according to the invention, can be treated as follows. An HCV antiviral compound can be administered to the patient, preferably in a biologically compatible solution or a pharmaceutically acceptable delivery vehicle, by ingestion, injection, inhalation or any number of other methods. The dosages administered will vary from patient to patient; a “therapeutically effective dose” can be determined, for example but not limited to, by the level of enhancement of function (e.g., inhibition of the formation of an eIF3/5′ UTR complex or inhibiting IRES dependent translation). Monitoring the level of inhibition of the formation of an eIF3/5′ UTR complex or inhibiting IRES dependent translation will also enable one skilled in the art to select and adjust the dosages administered. A dosage may be repeated daily, weekly, monthly, yearly, or as considered appropriate by the treating physician.

### Pharmaceutical Compositions

The invention provides for compositions comprising an HCV antiviral molecule, for example, identified in a screening assay according to the invention admixed with a physiologically compatible carrier. As used herein, “physiologically compatible carrier” refers to a physiologically acceptable diluent such as water, phosphate buffered saline, or saline, and further may include an adjuvant. Adjuvants such as incomplete Freund's adjuvant, aluminum phosphate, aluminum hydroxide, or alum are materials well known in the art.

The invention also provides for pharmaceutical compositions. In addition to the active ingredients, these pharmaceutical compositions may contain suitable pharmaceutically acceptable carrier preparations which can be used pharmaceutically.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. . . Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in a acceptable carrier have been prepared, they can be placed in an appropriate container and labelled for treatment of an indicated condition with information including amount, frequency and method of administration.

The invention further provides the use of the compounds of the invention in testing potential HCV antiviral molecules.

The above disclosure generally describes the present invention. A more complete understanding can be obtained by reference to the following specific examples, which are provided herein for purposes of illustration only and are not intended to limit the scope of the invention.

### EXAMPLES

### Example 1

### Identification of the mIRES

The effects of mutations in the IIIb loop in HCV were determined by IRES-dependent translation assay.

A striking reduction in IRES-dependent translation was noted when C at nucleotide 186 was changed to G, thereby creating a W-C base pair with nucleotide 211. Translation was only 39% of that driven by the wild-type sequence. Similarly, replacement of nucleotides 182 and 183 (AC) with CAUW, to form W.C base pairs with nucleotides 214 to 217, reduced translation to 24% of wild-type. Replacement of nucleotides 214 to 217 (AAUG) with GU, to form W.C base pairs with nucleotides 182 and 183, reduced translation to 34% of wild-type.

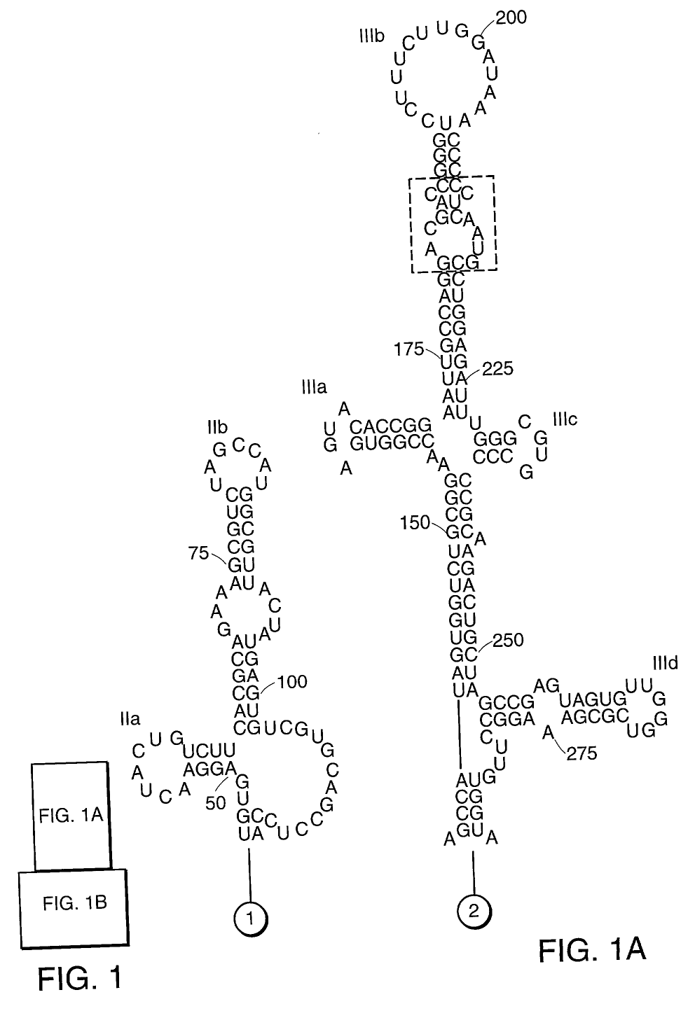

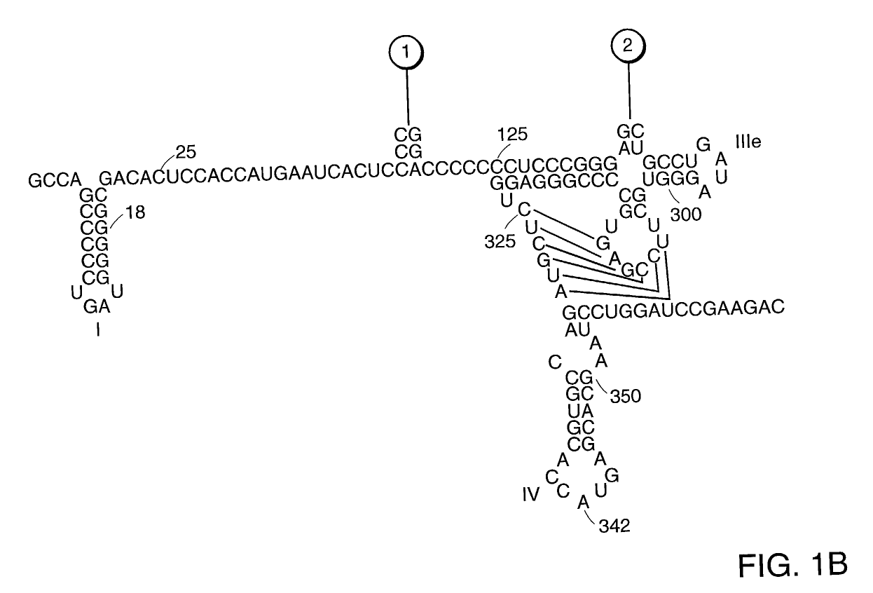

The two bulged regions boxed in FIG. 1 are thus critical for RES-driven translation. It is concluded that the binding interaction between eIF3 and the 5′-UTR in HCV genotype 1a requires the motif formed by the annealing of two short linear sequences 5′-G-A-C-G-A-C-C-3′ and 3′-C-G-U-A-A-C-U-C-G-5′. These anneal to form the mIRES.

It is surprising that two such short sequences are essential to the binding of eIF3 and can be used to mimic the full-length 5′-UTR. Firstly, interest has focused on domains IIIa and IIIc [iv], although some interactions with IIIb have been noted in crude footprinting studies [vi]. Secondly, the two short linear sequences are by no means specific for HCV. The 7mer sequence GACGACC is found in, for instance, human chromosome 19 (accession AC005602), pig Na-dependent glucose cotransporter (L02900), rat Id3a (AF000942, D10864), mouse helix-loop-helix protein (M60523), C. elegans collagen (Z22964), Halobacter NRC-1 plasmid (AF016485), and vWF from Bradypus tridactylus (U31603) and Chaetophractus villosus (AF076480). The 9mer sequence GCUCAAUGC is found in Xenopus laevis corticotropin releasing factor receptor (Y14036), human MHC1 (AF055066), Synechocystis PCC6803 (D90910), and mouse tenascin (D90343). Identity of the two mIRES constituent sequences from other HCV strains with non-HCV sequences is similarly prevalent. The two sequences individually, therefore, are not specific to HCV, but their juxtaposition in a three-dimensional annealed structure gives rise to an effective mimic of the native 5′-UTR.

### Example 2

### Mutation Analysis of the mIRES

To probe the critical residues within these 2 short regions, the mIRES was dissected internally.

The following single nucleotide mutations were made:

| Nucleotide | Wild-type residue | Mutation | Translation |
|---|---|---|---|
| 182 | A | C | 68% |
| | | G | 56% |
| | | U | 76% |
| 183 | C | A | 66% |
| | | G | 63% |
| | | U | 80% |
| 184 | G | A | 30% |
| | | U | 30% |
| 185 | A | U | 39% |
| | | G | 26% |
| | | C | 43% |
| 186 | C | G | 39% |
| 187 | C | G | 39% |
| 211 | C | A | 31% |
| | | G | 21% |
| | | U | 55% |
| 212 | U | A | 52% |
| | | G | 44% |
| | | C | 53% |
| 213 | C | A | 44% |
| | | G | 39% |
| | | U | 78% |
| 214 | A | C | 92% |
| | | G | 73% |
| | | U | 90% |
| 215 | A | G | 93% |
| | | U | 92% |
| 216 | U | C | 72% |
| | | G | 84% |
| 217 | G | A | 100% |
| | | C | 55% |
| | | U | 88% |

Pairs of mutations were also tested. The collected results were as follows:

| Nucleotide pair | Wild-type residues | Mutation | Translation |
|---|---|---|---|
| 182-217 | A-G | C-G | 68% |
| | | G-G | 56% |
| | | U-G | 76% |
| | | A-A | 100% |
| | | A-C | 55% |
| | | A-U | 88% |
| | | C-A | 66% |
| | | C-C | 33% |
| | | C-U | 43% |
| | | G-A | 97% |
| | | G-C | 72% |
| | | G-U | 89% |
| | | U-A | 44% |
| | | U-C | 61% |
| | | U-U | 63% |
| 183-214 | C-A | A-A | 66% |
| | | G-A | 63% |
| | | U-A | 80% |
| | | C-C | 92% |
| | | C-G | 73% |
| | | C-U | 90% |
| | | A-C | 79% |
| | | A-G | 40% |
| | | A-U | 89% |
| | | G-C | 91% |
| | | G-G | 74% |
| | | G-U | 90% |
| | | U-G | 86% |
| | | U-U | 78% |
| 184-213 | G-C | A-C | 30% |
| | | U-C | 30% |
| | | G-A | 44% |
| | | G-G | 39% |
| | | G-U | 78% |
| 185-212 | A-U | U-U | 39% |
| | | G-U | 26% |
| | | C-U | 43% |
| | | A-A | 52% |
| | | A-G | 44% |
| | | A-C | 53% |
| | | U-A | 90% |
| | | U-G | 43% |
| | | U-C | 45% |
| | | G-A | 49% |
| | | G-G | 27% |
| | | G-C | 34% |
| | | C-A | 54% |
| | | C-G | 71% |
| | | C-C | 45% |
| 186-211 | C-C | G-C | 39% |
| | | C-A | 31% |
| | | C-G | 21% |
| | | C-U | 55% |
| | | G-G | 35% |
| | | G-U | 30% |

One triple mutant was also tested (C¹⁸³ A+A²¹⁴ G+insertion of U at nucleotide 211). This reduced translation to 8% of wild-type.

From the above data, it is concluded that the mIRES will tolerate the following mutations whilst remaining functional:

| Nucleotide | Wild-type | Functional mutations |
|---|---|---|
| 182 | A | Can be mutated to C, G or U, provided that |
| | | when it is C, nucleotide 217 is not also C |
| 183 | C | Can be mutated to U, G or A, provided that |
| | | when it is A, nucleotide 214 is not G |
| 184 | G | None |
| 185 | A | Can be mutated to C, G or U, provided that (a) |
| | | nucleotide 212 is also mutated, and (b) when it |
| | | is G, nucleotide 212 is A. |
| 186 | C | None. |
| 211 | C | Can be mutated to U. |
| 212 | U | Can be mutated to A, C or G, provided that |
| | | when it is C or G, nucleotide 185 is not G. |
| 213 | C | Can be mutated to A, U or G. |
| 214 | A | Can be mutated to U, C or G, provided that, |
| | | when it is G, nucleotide 183 is not A. |
| 215 | A | Can be mutated to G or U or C |
| 216 | U | Can be mutated to C or G or A |
| 217 | G | Can be mutated to A, C or G, provided that |
| | | when it is C, residue 182 is not also C. |

### Example 3

### Analysis of the interaction between eIF3 and the HCV IRES

### Methods

The ability to interact with the HCV IRES was assessed with a RNA electrophoretic gel mobility shift assay (EMSA). The eIF3 complex was studied with the complete IRES RNA,

Domain III and deletions of Domain III. The ability of each probe to form a complex with eIF3 was determined by EMSA. In addition, to determine the relative affinity of each IRES RNA, competition analysis was performed with unlabelled RNA.

### Preparation of Radiolabelled IRES Probes and Unlabelled Competitors

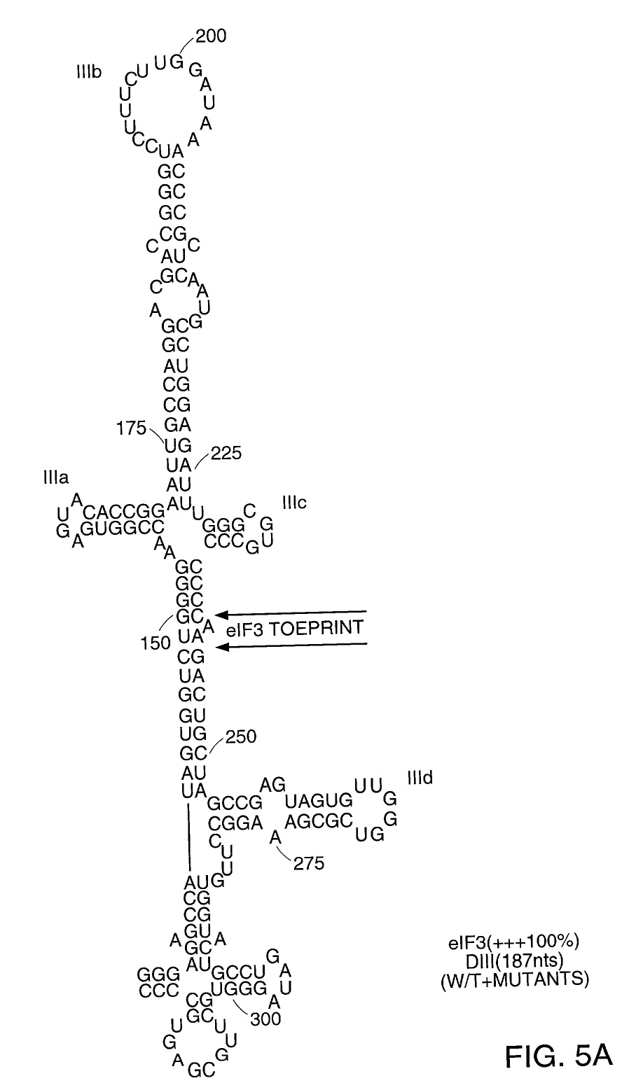

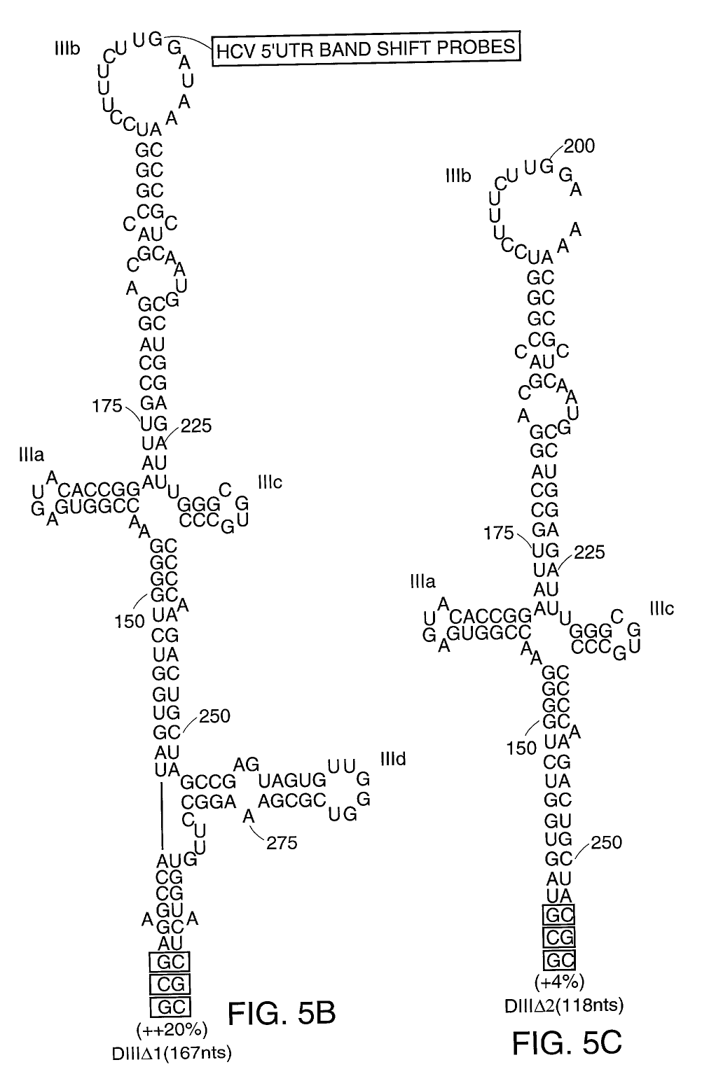

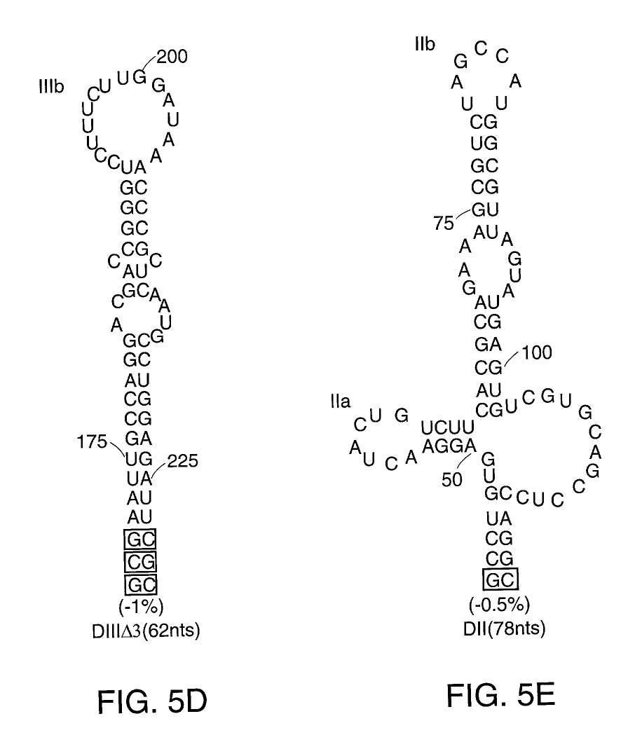

Plasmid templates (FIG. 5), linearised with EcoRI, were transcribed with T7 RNA polymerase in the presence of [32p]UTP or UTP and purified by denaturing electrophoresis and subsequent electroelution. The concentration of each prepared RNA was determined by UV spectroscopy and confirmed by denaturing electrophoresis.

### EMSA Binding Assay

IRES-eIF3 binding reactions were performed by incubating the radiolabelled probe (1 nM) with 0.01 μg of purified eIF3 on ice, in a total volume of 18 μl of binding buffer (60 mM KCl, 10 mM Hepes pH 7.4, 3.0 mM MgCl₂, 1 mM DTT, 5% glycerol and 200 nM tRNA). eIF3 is purified as described in Merrick, 1979, Methods in Enzymology, Vol LX, 101-108. The complex was incubated on ice for 10 min prior to electrophoresis. For competition experiments, unlabelled RNA was pre-incubated with eIF3 for 10 min prior to the addition of radiolabelled probe. The eIF3-IRES complex was resolved by non-denaturing electrophoresis. The gels were dried and quantified by phosphorimager analysis.

### Results

EMSA analysis clearly indicates that both the complete IRES and the isolated Domain III form a specific interaction with eIF3. Deletions of Domain III, DIIIΔ1 and DIIIΔ2, also form a specific complex with eIF3, albeit with reduced affinity. Further deletions of Domain III abolish complex formation. In addition, other regions of the IRES, such as Domain II both fail to bind eIF3 and demonstrate significant competition. This is also true for tRNA, an unrelated RNA. Deletions of Domain III with a relative affinity less than 4% (compared to Domain III) do not demonstrate selectivity (Table 1).

In summary, although the mIRES is critical for IRES activity and the interaction with eIF3, the mIRES is not sufficient for eIF3 binding. Other determinants of Domain III, such as domain IIIa/c are required for the interaction. In addition, the eIF3 EMSA system provides an assay to quantify the inhibitory activity of HCV IRES antiviral molecules or therapeutic compounds

TABLE 1
Binding properties of Domain III probes with eIF3

| | Ability to complex | Relative binding |
|---|---|---|
| RNA | with eIF3 | affinity |
| IRES (complete) | + | N/D |
| DIII | + | 100% |
| DIIIΔ1 | + | 20% |
| DIIIΔ2 | + | 4% |
| DIIIΔ3 | − | 1% |
| DII | − | 0.5% |
| tRNA | − | 0.1% |

### Example 4

### Fret Assay for the HCV Internal Loop (mIRES)

### Library of RNA-binding Peptides

To identify peptides binding to HCV IRES RNA, a library of peptides with a variable pentapeptide core and constant flanking neutral and basic amino acids was designed. All peptides contained the acceptor dye Dabsyl at the N-terminus. The three amino acids K, Q and R were used in the variable pentapetide part. From the possible 243 (3⁵) peptides a subset of 40 peptides was chosen for synthesis and testing (Table 2).

TABLE 2

| Library of RNA-binding peptides. | | | | | | | | | | | |
|---|---|---|---|---|---|---|---|---|---|---|---|
| The variable core is highlighted. | | | | | | | | | | | |
| No. | | | | | | | | | | | SEQ ID NO |
| 3 | N-Dabsyl | T | R | K | R | K | G | S | G | -Amide | 1 |
| 4 | N-Dabsyl | T | R | K | R | K | G | S | G | -Amide | 2 |
| 5 | N-Dabsyl | T | R | K | R | K | G | S | G | -Amide | 3 |
| 6 | N-Dabsyl | T | R | K | R | K | G | S | G | -Amide | 4 |
| 7 | N-Dabsyl | T | R | K | R | K | G | S | G | -Amide | 5 |
| 8 | N-Dabsyl | T | R | K | R | K | G | S | G | -Amide | 6 |
| 9 | N-Dabsyl | T | R | K | R | K | G | S | G | -Amide | 7 |
| 10 | N-Dabsyl | T | R | K | R | K | G | S | G | -Amide | 8 |
| 11 | N-Dabsyl | T | R | K | R | K | G | S | G | -Amide | 9 |
| 12 | N-Dabsyl | T | R | K | R | K | G | S | G | -Amide | 10 |
| 13 | N-Dabsyl | T | R | K | R | K | G | S | G | -Amide | 11 |
| 14 | N-Dabsyl | T | R | K | R | K | G | S | G | -Amide | 12 |
| 15 | N-Dabsyl | T | R | K | R | K | G | S | G | -Amide | 13 |
| 16 | N-Dabsyl | T | R | K | R | K | G | S | G | -Amide | 14 |
| 17 | N-Dabsyl | T | R | K | R | K | G | S | G | -Amide | 15 |
| 18 | N-Dabsyl | T | R | K | R | K | G | S | G | -Amide | 16 |
| 19 | N-Dabsyl | T | R | K | R | K | G | S | G | -Amide | 17 |
| 20 | N-Dabsyl | T | R | K | R | K | G | S | G | -Amide | 18 |
| 21 | N-Dabsyl | T | R | K | R | K | G | S | G | -Amide | 19 |
| 22 | N-Dabsyl | T | R | K | R | K | G | S | G | -Amide | 20 |
| 23 | N-Dabsyl | T | R | K | R | K | G | S | G | -Amide | 21 |
| 24 | N-Dabsyl | T | R | K | R | K | G | S | G | -Amide | 22 |
| 25 | N-Dabsyl | T | R | K | R | K | G | S | G | -Amide | 23 |
| 26 | N-Dabsyl | T | R | K | R | K | G | S | G | -Amide | 24 |
| 27 | N-Dabsyl | T | R | K | R | K | G | S | G | -Amide | 25 |
| 28 | N-Dabsyl | T | R | K | R | K | G | S | G | -Amide | 26 |
| 29 | N-Dabsyl | T | R | K | R | K | G | S | G | -Amide | 27 |
| 30 | N-Dabsyl | T | R | K | R | K | G | S | G | -Amide | 28 |
| 31 | N-Dabsyl | T | R | K | R | K | G | S | G | -Amide | 29 |
| 32 | N-Dabsyl | T | R | K | R | K | G | S | G | -Amide | 30 |
| 33 | N-Dabsyl | T | R | K | R | K | G | S | G | -Amide | 31 |
| 34 | N-Dabsyl | T | R | K | R | K | G | S | G | -Amide | 32 |
| 35 | N-Dabsyl | T | R | K | R | K | G | S | G | -Amide | 33 |
| 36 | N-Dabsyl | T | R | K | R | K | G | S | G | -Amide | 34 |
| 37 | N-Dabsyl | T | R | K | R | K | G | S | G | -Amide | 35 |
| 38 | N-Dabsyl | T | R | K | R | K | G | S | G | -Amide | 36 |
| 39 | N-Dabsyl | T | R | K | R | K | G | S | G | -Amide | 37 |
| 40 | N-Dabsyl | T | R | K | R | K | G | S | G | -Amide | 38 |
| 41 | N-Dabsyl | T | R | K | R | K | G | S | G | -Amide | 39 |
| 42 | N-Dabsyl | T | R | K | R | K | G | S | G | -Amide | 40 |

### Fluorescein-labeled RNAs

Oligoribonucleotides with a fluorescein attached to the 5′ end of the molecule (shown in FIG. 6a) were synthesised using conventional oligoribonucleotide techniques. A 19 mer (FIG. 6b) was used for control purposes

### Measurement of RNA/Peptide Interaction by FRET

Fluoresein-labeled RNA was titrated with Dabsyl-labelled peptides. The donor (fluorescein) fluoresence was excited at 490 nm. In the presence of peptide, binding leads to a quenching of fluorescence by the non-fluorescent acceptor dye Dabsyl. The quenching curves allow the determination of binding affinities. Fluorescein-labelled RNA containing the mIRES was titrated with various peptides.

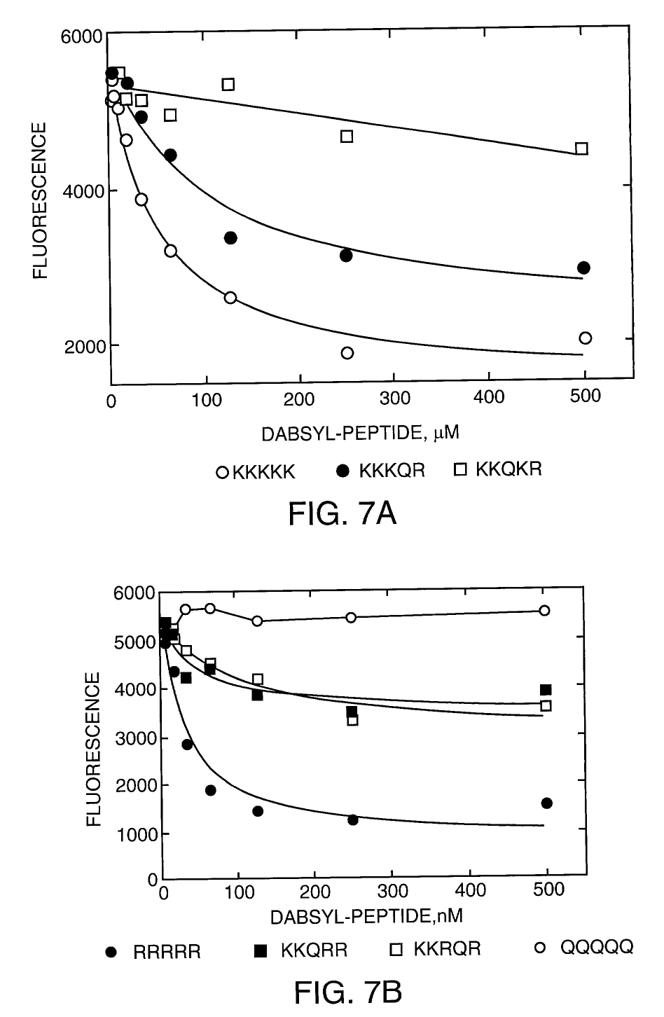

FIG. 7 illustrates binding curves for RNA containing the mIRES titrated with various peptides. Experimental conditions: Fam-labelled RNA (10 nM) was titrated with Dabsyl-labelled peptide in 50 mM Tris/Cl, pH7.4, 110 mM KCl, 5ug/ml BSA, 0.01% Triton X-100, 1%DMSO. Fluorescence (excitation 490 nm/emission at 535 nm) was determined in 96-well plates using a Fluorescence plate reader. The assay volume was 100 μl.

### Binding Data for Various Peptides to HCV mIRES RNA

Dissociation constants were determined by fitting a binding isotherm (1-site-binding) to the titration data using the equation

Y=((Fmax−Fmin)−((Fmax−Fmin)*c/(Kd+c)))+Fmin

wherein

Fmax: fluorescence in the absence of acceptor

Fmin: fluorescence at saturation

c: peptide conc.

The FRET based displacement assay of the invention with a nM peptide ligand for the HCV mIRES identified herein provides a primary screen for compounds targeted to the internal loop that can disrupt the eIF3-5′-UTR interaction.

TABLE 3
Affinities of various peptides
for HCV mIRES RNA.

| RNA | Peptide | Kd(nM) | R² | SEQ ID NO |
|---|---|---|---|---|
| HCV | RRRRR | 30 ± 10 | 0.96 | 41 |
| loopIII | KKQRR | 41 | 0.91 | 42 |
| | KKKKK | 49 | 0.98 | 43 |
| | KKKQR | 90 | 0.93 | 44 |
| | KKRQR | 104 | 0.95 | 45 |
| | KKQKR | >500 | 0.99 | 46 |
| | QQQQQ | >500 | N/A | 47 |
| 19mer | RRRRR | 194 | 0.97 | 42 |
| control | KKRQR | >500 | N/A | 46 |
| RNA | KKQKR | >500 | N/A | 47 |
| | KKKKK | >500 | N/A | 44 |
| | KKQRR | >500 | N/A | 43 |
| | KKKQR | >500 | N/A | 45 |
| | QQQQQ | >500 | N/A | 48 |

### Example 5

### Binding of Paromomycin-TAMRA to HCV IIIB by FRET

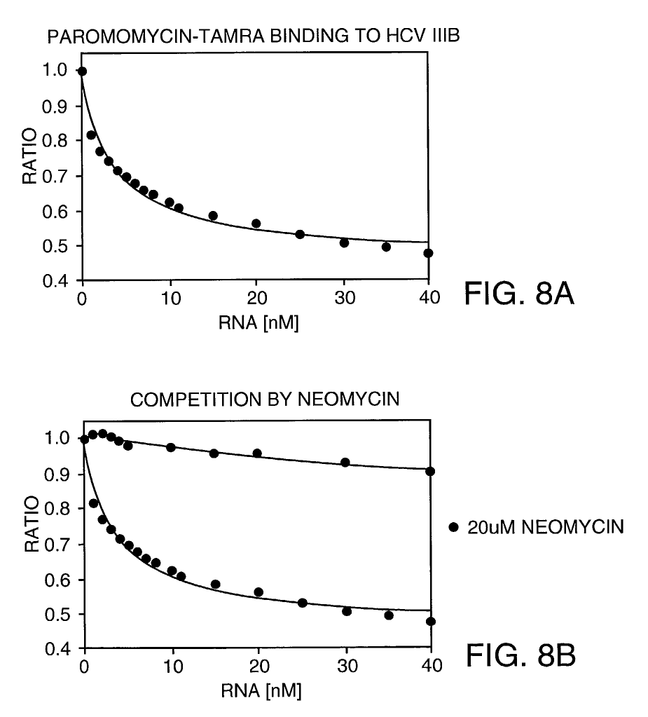

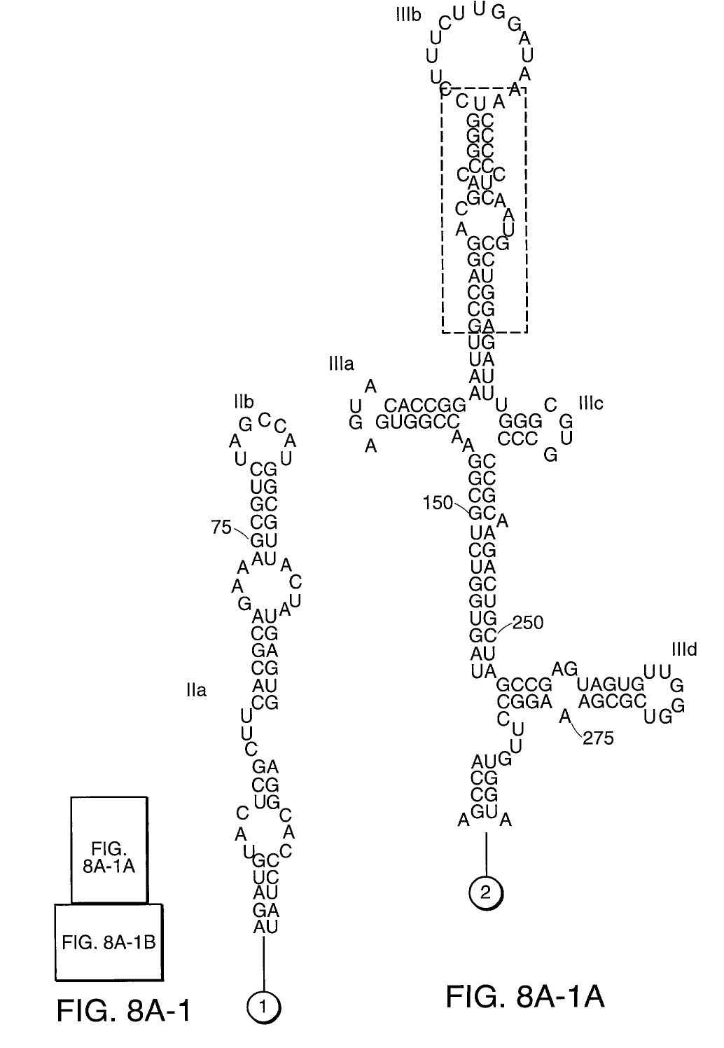

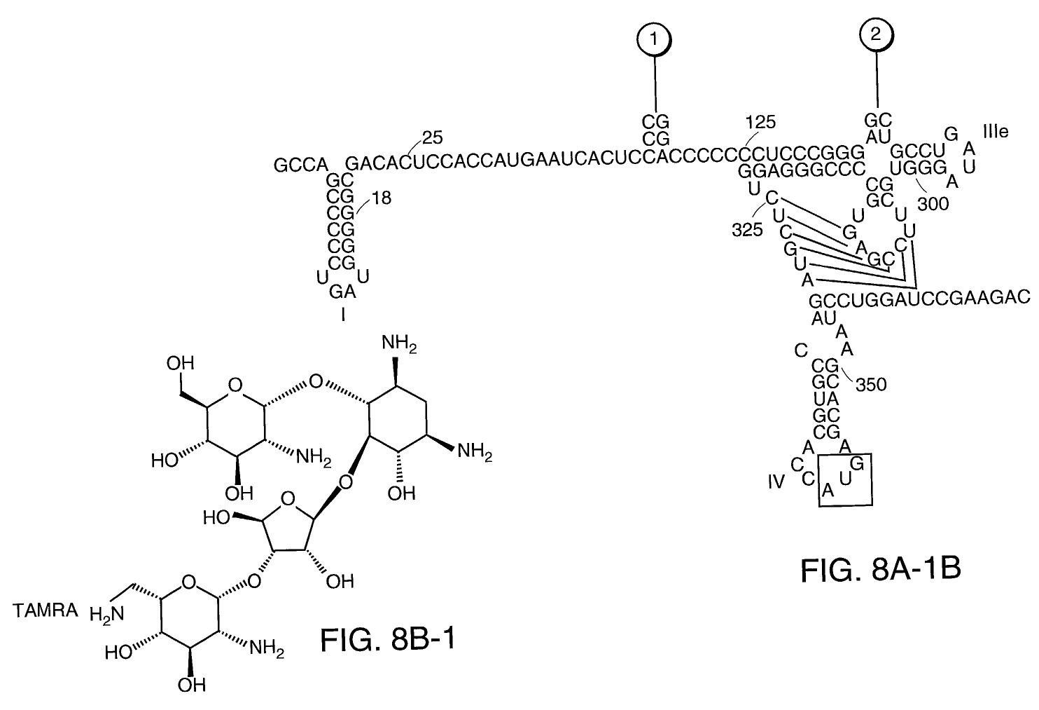

Paromomycin-TAMRA binding to an HCV derived RNA fragment corresponding to IIIB-Dabcyl RNA was demonstrated by titrating IIIB RNA in the presence of 10 nM paromomycin as shown in FIG. 8A. When Paromomycin-TAMRA binds to IIIB-Dabcyl RNA, fluorescence resonance energy transfer (FRET) can take place between the fluorescent donor (Tetramethyl rhodamine (TAMRA)) and the non-fluorescent acceptor (dabcyl). When FRET takes place, it is observed as a reduction in fluorescent donor emission, and a concomitant increase in the fluorescent acceptor emission. The dabcyl group is not fluorescent. In this experiment FRET is measured by the reduction in donor (TAMRA) fluorescence.

### Synthesis of Paramomycin-TAMRA

Paramomycin-TAMRA was synthesized by reacting 55 mg paramomycin sulphate in sodium bicarbonate (6 mL 0.067M in 30% dimethyl formamide (DMF)) with 5 mg 5-carboxytetramethyl rhodamine (in 1 mL DMF) over 12 hours at room temp. The solution was diluted and purified by anion exchange chromatography, and reversed phase HPLC (ix).

### RNA Synthesis and Sequence

RNA was synthesised by solid phase methods. A 3′ Dabcyl group was incorporated as a non-fluorescent acceptor. The sequence of the RNA is:

| | |
|---|---|
| 5′-GGG ACG ACC GCU UCG GCG CUC AAU GCC C- Dabcyl 3′ | (SEQ ID NO:41) |

The interaction between paromomycin-TAMRA and IIIB-Dabcyl RNA was measured utilising paromomycin-TAMRA as a donor and IIIB-Dabcyl RNA as an acceptor. Each measurement was made in a 2 mL cuvette. Increasing amounts of IIIB-Dabcyl RNA (corresponding to the amounts shown in FIG. 8a) were added to a solution of 10 nM paromomycin-TAMRA in the presence of 50 mM Tris.HCl pH7.5, 80 mM KCl. For each titration point, emission spectra were acquired using a fixed wavelength of 552 nm with the excitation slits set to 5 nm and the emission slits set to 10 nm. Emission spectra were acquired over the range 570-600 nm. This range encompasses the emission spectrum of the donor (tetramethyl rhodamine). A reduction in donor intensity was observed, due to FRET taking place between the two dyes upon peptide binding to the IIIB- RNA. The K_{d} for binding is 9.3 (+/−1.5) nM for the binding of paromomycin-TAMRA to the IIIB RNA. The donor ratio presented is the difference in donor intensity on addition of IIIB-RNA as a proportion of the total donor intensity in the absence of acceptor.

The reduction in TAMRA fluorescence in the presence of increasing amounts of RNA is consistent with FRET taking place between the TAMRA and dabcyl groups. This is consistent with paromomycin-TAMRA binding to IIIB RNA.

### Example 6

### Inhibition of Paromomycin-TAMRA Binding to IIIB-Dabcyl RNA by Neomycin

Neomycin (10 uM) inhibits paromomycin-TAMRA binding to IIIB-Dabcyl RNA. Inhibition of Paromomycin-TAMRA binding to IIIB-Dabcyl RNA was demonstrated by titrating IIIB RNA with 10 nM paromomycin-TAMRA in the presence of 10 uM neomycin. The results are shown in FIG. 8B.

Measurements were made in a 2 mL cuvette. Increasing amounts of IIIB-Dabcyl RNA (corresponding to the amounts shown in the figure) were added to a solution of 10 nM paromomycin-TAMRA in the presence of 50 mM Tris.HCl pH7.5, 80 mM KCl. For each titration point emission spectra were acquired using a fixed wavelength of 552 nm with the excitation slits set to 5 nm and the emission slits set to 10 nm. Emission spectra were acquired over the range 570-600 nm. Two binding curves are presented, the first in the absence of a test molecule (FIG. 8a) and the second in the presence of 10 μM neomycin (FIG. 8b). The presence of neomycin prevents binding of paromomycin-TAMRA to IIIB RNA and the reduction in donor signal due to FRET does not occur. Neomycin inhibits paromomycin-TAMRA to IIIB RNA binding with a Ki of 520 (+/−33) nM.

In this experiment FRET between the paromomycin-TAMRA and the IIIBDabcyl-RNA does not take place in the presence of neomycin so there is no reduction in TAMRA fluorescence in the presence of RNA.

It will be understood that the invention is described above by way of example only and modifications may be made whilst remaining within the scope and spirit of the invention.

### Other Embodiments

Other Embodiments are within the claims that follow.

i. Das et al. (1998) Frontiers in Bioscience 3:d1241-1252.

ii. Das et al. (1998) J. Virol. 72:5638-5647.

iii. Hanecak et al. (1996) J. Virol 70:5203-5212.

iv. Tang et al. (1999) J. Virol. 73:2359-2364

v. Collier et al. (1998) J. Virol. 79:2359-2366.

vi. Sizova et al. (1998) J. Virol. 72:4775-4782.

vii. Kieft et al. (1999) J. Mol. Biol. 292(3):513-529.

viii. Buratti et al. (1998) Nucleic Acid Research 26(13):3179-3187.

ix. (1997) Biochemistry (36): 768-779.

1. A compound comprising nucleotide sequences:
5′-P-N¹-N²-G-N³-C-I-3′ and 3′-Q-N⁴-N⁵-N⁶-N⁷N⁸-N⁹-Y-J-5′
wherein
P and Q are any two nucleotides that can form a Watson-Crick base pair,
I and J are any two nucleotides that can form a base pair,
N¹ and N⁴ are not both C,
when N² is A, N⁷ is not G,
N⁹ can only be U if N³ is A, and
when N³ is G, N⁹ is A;
and said sequences can anneal to each other, and said compound comprises 200 or fewer nucleotides.

2. A compound comprising nucleotide sequences:
5′-P-N¹-N²-G-N³-C-I-3′ and 3′-Q-N⁴-N⁵-N⁶-N⁷-N⁸-N⁹-Y-J-5′
wherein
P and Q are any two nucleotides that can form a Watson-Crick base pair,
I and J are any two nucleotides that can form a base pair,
N¹ and N⁴ are not both C,
when N² is A, N⁷ is not G,
N⁹ can only be U if N³ is A, and
when N³is G, N⁹ is A;
and said sequences can anneal to each other, and said compound is not a complete HCV genome or fragment thereof.

3. The compound of claim 1 or 2 wherein the 3′ end of the sequence 5′-P-N¹-N²-G-N³-C-I-3′ and the 5′ end of the sequence 3′-Q-N⁴-N⁵-N⁶-N⁷-N⁸-N⁹-Y-J-5′ are joined by which allows said sequences to anneal to each other.

4. The compound of claim 3 comprising the sequence
5′-(N)ₐ-[P-N-¹-N²-G-N³-C-I]-(N)_{b}-[J-Y-N⁹-N⁸-N⁷-N⁶-N⁵-N⁴-Q]-(N)_{c}-3′
wherein N may or may not be identical and is any nucleotide,
a is zero or an integer from 1 to 100,
b is an integer from 3 to 100, and
c is zero or an integer from 1 to 100, and
said linker is —(N)_{b}—.

5. The compound of claim 4 wherein linker —(N)_{b}— is a stabilising sequence.

6. The compound of claim 4 wherein said linker —(N)_{b}— comprises a loop.

7. The compound of claim 6 wherein said linker —(N)_{b}— comprises the structure: 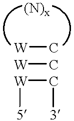
wherein x is an integer from 4 to 20, and wherein W-C is a Watson-Crick base Pair, and each W-C may or may not be identical.

8. A compound comprising a first nucleic acid strand comprising the sequence 5′-P-N¹-N²-G-N³-C-I-3′ annealed to a second nucleic acid strand comprising the sequence 3′-Q-N⁴-N⁵-N⁶-N⁷-N⁸-N⁹-Y-J-5′, wherein
(a) P and Q are any two nucleotides that can form a Watson-Crick base pair,
(b) I and J are any two nucleotides that can form a base pair,
(c) N¹ and N⁴ are not both C,
(d) when N² is A, N⁷ is not G,
(e) N⁹ can only be U if N³ is A, and
(f) when N³ is G, N⁹ is A.

9. The compound of claim 8 wherein I and J form a Watson-Crick base pair.

10. A compound comprising a first nucleic acid strand comprising the sequence 5′ (N)ₐ-P-N¹-N²-G-N³-C-I-(N)_{b}_{¹}-3′ annealed to a second nucleic acid strand comprising the sequence 3′-(N)_{c}-Q-N⁴-N⁵-N⁶-N⁷-N⁸-N⁹-Y-J-(N)_{b}_{²}-5′, wherein
a is zero or an integer from 1 to 100,
b¹ is zero or an integer from 1 to 100,
b² is zero or an integer from 1 to 100, and
c is zero or an integer from 1 to 100.

11. The compound of claim 10 wherein the combination of (N)_{b1} and (N)_{b2} comprise a stabilising group, or (N)_{b1} and (N)_{b2} each, independently comprise a stabilising group.

12. The compound of claim 10 or 11 wherein —(N)_{b1}— and (N)_{b2} can anneal to each other.

13. The compound of claim 4 wherein the combination of —(N)ₐ— and —(N)_{c}— comprise a stabilising group, or —(N)ₐ— and —(N)_{c}— each, independently comprise a stabilising group.

14. The compound of claims 10 wherein the combination of —(N)ₐ— and —(N)_{c}— comprise a stabilising group, or —(N)ₐ— and —(N)_{c}— each, independently comprise a stabilising group.

15. The compound of claims 4 wherein —(N)ₐ— and —(N)_{c}— can anneal to each other.

16. The compound of claim 10 wherein —(N)ₐ— and —(N)_{c}— can anneal to each other.

17. The compound of claim 13 wherein —(N)ₐ— and —(N)_{c}— are capable of forming a structure comprising: 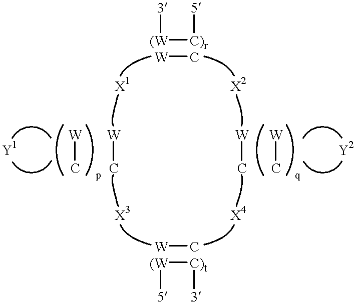
wherein each W.C may or may not be identical and is a Watson-Crick base pair,
X¹, X², X³ and X⁴ may or may not be identical and each, independently comprise from zero to four nucleotides,
Y¹ and Y² may or may not be identical and each, independently comprise from three to ten nucleotides, and
p, q, r and t may or may not be identical and each, independently comprise an integer from zero to ten.

18. The compound of claim 14 wherein —(N)ₐ— and —(N)_{c}— are capable of forming a structure comprising: 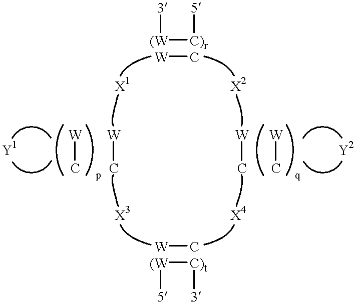
wherein each W.C may or may not be identical and is a Watson-Crick base pair,
X¹, X², X³ and X⁴ may or may not be identical and each, independently comprise from zero to four nucleotides,
Y¹ and Y² may or may not be identical and each, independently comprise from three to ten nucleotides, and
p, q, r and t may or may not be identical and each, independently comprise an integer from zero to ten.

19. The compound of claim 13 wherein —(N)ₐ— and —(N)_{c}— are capable of forming a structure comprising:

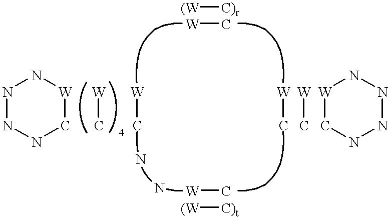

20. The compound of claim 14 wherein —(N)ₐ— and —(N)_{c}— are capable of forming a structure comprising:

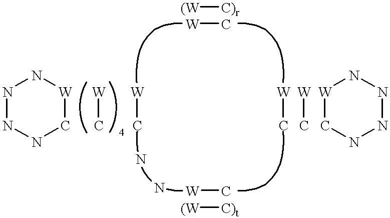

21. The compound of claim 1, 2, 8 or 10, wherein P is a purine; Q is a pyrimidine; I is a pyrimidine; J is a purine; N¹ is A, G or U; N² is A, C or U; N³ is A; N⁴ is a purine; N⁵ is U; N⁶ is a purine; N⁷ is A,G or U; N⁸ is C; N⁹ is U; and Y is C.

22. The compound of claim 1, 2, 8 or 10 further comprising modified nucleotide bases and/or modified sugars and/or modified linkages.

23. An assay, comprising the steps of (a) incubating a test molecule with the compound of claim 1, 2, 8 or 10, and (b) detecting the formation of a binding complex comprising said test molecule and said compound.

24. An assay comprising the steps of (a) incubating a test molecule with the compound of claim 1, 2, 8 or 10, and a ligand that binds specifically to said compound, and (b) determining the amount of a complex comprising said compound and said ligand.

25. An assay comprising the steps of: (a) contacting a test molecule with a pair of indicator molecules comprising (i) a ligand that is a labelled reporter and (ii) the compound of claim 1, 2, 8 or 10, wherein each member of said pair of indicator molecules is independently labelled with a complementary acceptor or donor group, said pair of indicator molecules binds specifically to each other in an orientation that permits the donor group to come into sufficient proximity to the acceptor group to permit fluorescent resonance energy transfer and/or quenching to take place; and (b) measuring the fluorescence of said compound and/or said ligand in the presence of the test molecule and, (c) comparing this value to the fluorescence of at least one standard.

26. An assay according to claim 23, wherein the assay is a screening assay to detect potential HCV antiviral molecules.

27. An assay according to claim 24, wherein the assay is a screening assay to detect potential HCV antiviral molecules.

28. An assay according to claim 25, wherein the assay is a screening assay to detect potential HCV antiviral molecules.

29. An assay according to claim 23, wherein said test molecule is an HCV antiviral molecule.

30. An assay according to claim 24, wherein said test molecule is an HCV antiviral molecule.

31. An assay according to claim 25, wherein said test molecule is an HCV antiviral molecule.

32. A kit for determining whether a test molecule binds specifically to the compound of claim 1, 2, 8 or 10, said kit comprising (a) the compound of claim 1, 2, 8 or 10 and (b) a ligand that binds specifically to said compound, wherein either or both said ligand and said compound are labelled.

33. A kit comprising (a) the compound of claim 1, 2, 8 or 10 labelled with a donor group or an acceptor group and (b) a ligand reporter labelled with a complementary acceptor or donor group, wherein said ligand reporter and said compound bind specifically to each other in an orientation that permits the donor group to come into sufficient proximity to the acceptor group to permit fluorescent resonance energy transfer and/or quenching.
